(19) United States
(12) Patent Application Publication
(10) Pub. No.: US 2021/0202033 A1
(43) Pub. Date: Jul. 1, 2021

(54) NOVEL MACHINE LEARNING APPROACH FOR THE IDENTIFICATION OF GENOMIC FEATURES ASSOCIATED WITH EPIGENETIC CONTROL REGIONS AND TRANSGENERATIONAL INHERITANCE OF EPIMUTATIONS

(71) Applicant: Washington State University, Pullman, WA (US)

(72) Inventors: Michael K. Skinner, Pullman, WA (US); Md. Muksitul Haque, Pullman, WA (US)

(21) Appl. No.: 16/888,922

(22) Filed: Jun. 1, 2020

Related U.S. Application Data
(63) Continuation of application No. 15/343,516, filed on Nov. 4, 2016, now abandoned.
(60) Provisional application No. 62/252,600, filed on Nov. 9, 2015.

(51) Int. Cl. G16B 30/00; G16B 40/00

(52) U.S. Cl. CPC G16B 30/00; G16B 40/00

(57) ABSTRACT

A two-step (sequential) machine learning analysis tool is provided that involves a combination of an initial active learning step followed by an imbalance class learner (ACL-ICL) protocol. This technique provides a more tightly integrated approach for a more efficient and accurate machine learning analysis. The combination of ACL and ICL work synergistically to improve the accuracy and efficiency of machine learning and can be used with any type of dataset including biological datasets.

### BACKGROUND OF THE INVENTION

### Field of the Invention

The invention generally relates to the identification of epigenetic modification and/or epigenetic regulatory regions of DNA that are associated with the transgenerational inheritance of epimutations using a sequential machine learning approach. In particular, the invention provides the sequential application of Active Learning analysis and Imbalance Class Learner analysis to epigenetic datasets.

### Background of the Invention

The current paradigm for the etiology of heritable diseases, including those caused by environmental insult, is based primarily on mechanisms of genetic alterations such as DNA sequence mutations. However, the majority of inherited diseases have not been linked to specific genetic abnormalities or changes in DNA sequence. In addition, the majority of environmental factors known to cause or influence the development of disease—including heritable diseases—do not have the capacity to alter DNA sequence. Therefore, additional molecular mechanisms need to be taken into account when attempting to clarify the etiology of diseases and to develop diagnostic tools and treatments.

Epigenetics is defined as “molecular factors and processes around DNA that regulate genome activity independent of DNA sequence and are mitotically stable” [1]. The molecular factors currently known to be epigenetic processes include DNA methylation, histone modifications, chromatin structure and selected non-coding RNA [1,3-7]. Epigenetics has been shown to be a critical factor in normal biology, disease etiology and evolution [1,8]. A combination of epigenetic and genetic molecular mechanisms will be essential for nearly all biological processes. However, genetics has been the primary molecular component considered for nearly all aspects of biology. For example, DNA sequence and genetics has been considered the primary form of inheritance. More recently, environmentally induced epigenetic transgenerational inheritance has been described in species from plants to humans [1]. This provides an additional epigenetic mechanism for inheritance to consider [9] and helps explain forms of familial inheritance not easily explained with classical genetics.

Epigenetic transgenerational inheritance is defined as “germline transmission of epigenetic information between generations in the absence of direct environmental exposure” [1]. A growing number of environmental factors have been shown to promote the epigenetic transgenerational inheritance of disease and phenotypic variation from nutrition, stress or toxicants [1,10]. The environmental chemicals shown to promote transgenerational inheritance of disease and sperm epimutations include the agricultural fungicide vinclozolin [1], pesticide permethrin and insect repellent N,N-diethyl-meta-toluamide (DEET) [12], pesticides methoxychlor [13] and dichlorodiphenyltrichloroethane (DDT) [14], plastic derived compounds bisphenol A (BPA) and phthalates [15], and hydrocarbon mixtures (jet fuel, JP8) [16]. The F0 generation gestating female rats were transiently exposed during fetal gonadal development and then the F1, F2 and F3 generations generated [1,11]. The transgenerational F3 generation (i.e., no direct exposure) was found to have a large number of high frequency disease states including testis, ovary, prostate, mammary and kidney disease [17].

Analysis of the F3 generation male sperm demonstrated differential DNA methylation regions (DMRs) that were highly reproducible and exposure specific [18,19]. These DMRs were termed epimutations and ranged in number for genome-wide promoter regions from 30 to 300 depending on the specific exposure [13,14,18]. Each transgenerational set of epimutations was found to be exposure specific with negligible overlap between exposures [1,18]. In addition to the transgenerational sperm epimutations, somatic cell transgenerational epimutations for the agricultural vinclozolin lineage F3 generation testicular Sertoli cells and ovarian granulosa cells were utilized in a similar analysis [20,21]. As found with the exposure specific sperm epimutations, the somatic cell epimutation sets were cell specific with negligible overlap. These somatic cell transgenerational epimutation data sets were also used independently in the current study as training sets for machine learning predictions for somatic cells versus germ cells.

These transgenerational epimutations were used to identify common genomic features associated with the epimutations. The first genomic feature found associated with all epimutations [18] was a low CpG density of less than 10 CpG per 100 bp which were characterized as “CpG deserts” containing small CpG clusters with differential DNA methylation [22] (see also U.S. Patent Publication 2013/0226468 to Skinner et al. herein incorporated by reference). The second set of genomic features identified were unique DNA sequences generally within a few hundred base pair of the differential DNA methylation region [23]. These DNA sequence motifs were previously shown to associate with binding proteins that bend DNA [19,23]. In addition to these genomic features, a number of other genomic features previously shown to associate with epigenetic sites were also selected for the analysis [24].

Despite the various genomic features identified to date, improved genome-wide methods of identifying epigenetic modification and/or epigenetic regulatory regions of DNA that are associated with the transgenerational inheritance of epimutations are urgently needed.

### SUMMARY OF THE INVENTION

Aspects of the present invention provide a novel machine learning approach to further identify the genomic features of the transgenerational germline epimutations and predict genome-wide sites that may be susceptible to become environmentally modified epimutations.

One aspect of the invention provides a computer-implemented method of identifying potential genomic locations and regulatory sites of epimutations, comprising inputting into a computer at least one genomic DNA sequence; identifying, with said computer, one or more regions of said at least one genomic DNA sequence which comprise one or both of potential locations of epimutations and potential regulatory sites of epimutations by a) training the computer with at least one training set comprising known epimutations to determine a set of potential genomic features associated with the known epimutations; b) using the trained computer to perform Active Learning analysis to identify the optimal genomic features from the set of potential genomic features that allow for the identification of the known epimutations in the training sets; c) using Imbalance Class Learner analysis to correct for data set imbalance; and d) selecting one or more regions in the genomic DNA sequence that contains one or more of the identified optimal genomic features; wherein said one or more regions comprise one or both of potential locations of epimutations and potential regulatory sites of epimutations and wherein said steps b) and c) are performed sequentially or simultaneously.

In some embodiments, steps a)-d) are performed on a server operationally connected to said computer. In some embodiments, the genomic DNA sequence is obtained from a nucleotide sequencing apparatus that is operationally linked to said computer. In other embodiments, the genomic DNA sequence is obtained from a second computer containing a database of genomic DNA sequences. In some embodiments, the computer-implemented method further comprises the step of, with said computer, identifying, within said one or more regions of said at least one genomic DNA sequence, at least one DNA sequence motif that is associated with one or both of epimutations and regulatory sites of epimutations.

Another aspect of the invention provides a system comprising i) a computer; ii) at least one non-transient storage medium comprising computer executable instructions which are performed by said computer and which cause said computer to carry out the steps of a) receiving at least one genomic DNA sequence as input; b) training with at least one training set comprising known epimutations to determine a set of potential genomic features associated with the known epimutations; c) performing Active Learning analysis to identify the optimal genomic features from the set of potential genomic features that allow for the identification of the known epimutations in the training sets; d) using Imbalance Class Learner analysis to correct for data set imbalance; and e) selecting one or more regions in the genomic DNA sequence that contains one or more of the identified optimal genomic features; wherein said steps c) and d) are performed sequentially or simultaneously; and iii) an output device capable of presenting results obtained by said computer in said selecting step.

In some embodiments, the system further comprises a server wherein said computer executable instructions which are performed by said computer cause said computer to carry out steps b) and e) on said server. In some embodiments, the system further comprises a nucleotide sequencing apparatus wherein said at least one non-transient storage medium further comprises instructions for causing said computer to receive said at least one genomic DNA sequence from said nucleotide sequencing apparatus. In some embodiments, the system further comprises a second computer containing a database of genomic DNA sequences wherein said at least one non-transient storage medium further comprises instructions for causing said computer to receive said at least one genomic DNA sequence from said database on the second computer. In some embodiments, the output device is selected from the group consisting of a printer, display, and modem.

Another aspect of the invention provides a method for the early intervention and treatment of a subject who is suspected of or who has been exposed to an environmental agent or who has or is suspected of having a disease or condition of interest, comprising inputting into a computer at least one genomic DNA sequence from said subject and from a positive control; identifying, with said computer, one or more regions of said at least one genomic DNA sequence which comprise one or both of potential locations of epimutations and potential regulatory sites of epimutations by a) training the computer with at least one training set comprising known epimutations to determine a set of potential genomic features associated with the known epimutations; b) using the trained computer to perform Active Learning analysis to identify the optimal genomic features from the set of potential genomic features that allow for the identification of the known epimutations in the training sets; c) using Imbalance Class Learner analysis to correct for data set imbalance; and d) selecting one or more regions in the genomic DNA sequence that contains one or more of the identified optimal genomic features; wherein said one or more regions comprise one or both of potential locations of epimutations and potential regulatory sites of epimutations and wherein said steps b) and c) are performed sequentially or simultaneously; determining the presence or absence of an epigenetic modification within said one or more regions of genomic DNA in said subject and said positive control; comparing the epimutations of said one or more regions of the positive control to the same one or more regions in a genomic DNA sequence of the subject; and administering an appropriate treatment protocol to said subject if said one or more regions of the genomic DNA sequence of the subject contains epigenetic mutations in the same locations as the positive control.

In some embodiments, the environmental agent is selected from the group consisting of vinclozolin, dioxin, permethrin, N,N-diethyl-meta-toluamide (DEET), methoxychlor, dichlorodiphenyltrichloroethane (DDT), bisphenol A (BPA), phthalates, and hydrocarbon jet fuel. In some embodiments, the disease or condition is selected from the group consisting of low sperm production, abnormalities of sexual organs, ovarian cysts, kidney abnormalities, prostate disease, and immune abnormalities.

### BRIEF DESCRIPTION OF THE DRAWINGS

FIG. 1. Machine learning approach and training set description. Flow chart of two-step machine learning framework for DMR identification.

FIG. 2. Schematic representation of an exemplary computerized system of the invention.

FIG. 3. Flow chart of ACL.

FIG. 4. Chromosomal plot of germ cell dataset DHVPP shows the predicted 3+ sites and the clusters. Predicted potential DMR sites (3,233) when DHVPP is used as the training set with lines in the bottom and clusters (80) with boxes on the top for each chromosome line. X-axis shows each of the 21 chromosomes while Y-axis shows the length of the chromosome with predicted potential DMR locations. The clusters are regions which indicate over-representations of the sites within the small sub-section of the genome.

FIG. 5. Chromosomal plot of somatic cell dataset SG shows the predicted 3+ sites and the clusters. Potential predicted DMR sites (1,503) when SG is used as the training set to predict on the rest of the genome. X-axis shows each of the 21 chromosomes while Y-axis shows the length of the chromosome with predicted potential DMR locations. Lines in the bottom are shown as potential DMR sites and clusters (44) with boxes are shown on the top of each chromosome.

FIG. 6A-B. CpG density plot showing number of predicted DMR sites correlated with CpG density. (A) CpG density from the potential predicted germ cell DMR sites (3,234) when DHVPP is used as the training set to predict genome-wide. (B) CpG density from the potential predicted somatic cell DMR sites (1,502) when SG is used as the training set to predict genome-wide. X-axis shows the number of CpG's per 100 bases on average while Y-axis shows the number of sites.

FIG. 7A-B. Predictive power of specific features. (A) Groups of features with their predictive power (percent accuracy) for the DHVPP dataset. (B) Groups of features with the predictive power (percent accuracy) for the SG dataset. The features include RE—Repeat Elements, TF—Transcription Factors, SM—Sequence Motifs, MM—Mammalian Motifs with their predictive power indicated.

FIG. 8A-B. Predictive power of repeat elements accuracy based on genomic location of 1 k, 5 k, 100 k from the DMR. (A) Combined average when each group of repeat elements are used for prediction for DHVPP dataset. (B) Combined average when each group of repeat elements are used for prediction for SG dataset. Shows combined repeat elements in the 100 k, 5 k and 1 k upstream and downstream regions.

FIG. 9A-B. Overlap between germ cell and somatic cell predicted sites. (A) Overlap between predicted DMR (+3 sites) from the two different datasets. (B) Overlap between predicted DMR (single sites) from the two different datasets.

FIG. 10. Overlap of germ cell validation set MXC-DDT with predicted DHVPP single probe data set.

### DETAILED DESCRIPTION

Many diseases, even those which are passed from parent to offspring, are not caused by genetic mutations. Rather, the causes of these diseases can be traced to epigenetic modifications of the genome. Aspects of the invention provide methods of identifying regions of DNA which are likely to harbor and/or regulate such epigenetic modifications using machine learning analysis.

A machine learning analysis uses a known training set(s) of data to construct a classifier based on known features to classify larger unknown data sets. Generally an issue with machine learning analysis is that a relatively small set of positive traits are used in reference to a much larger set (i.e., volume) of data with negative (non-relevant) traits. This introduces significant bias in the results due to the imbalance between data sets. In addition, often large sets of predicted features are used in machine learning analysis such that only a small number of critical features are relevant. This can also reduce the efficiency and bias the machine learning analysis.

Aspects of the present invention provide two different machine learning techniques to address these issues. Active learning (ACL) is the selection of important features and examples for an Oracle (e.g. a human expert) to classify. The addition of generalized query to the ACL allows selection of the optimal features in these examples which the Oracle can classify. The Oracle uses the optimal features identified by ACL, to then do imbalance learning and eventually the prediction. ACL can also be used to select the most important features and provide insights into the critical features identified. Imbalance class learners (ICL) can be used to reduce the data set imbalance bias and allow for a more accurate analysis. These two techniques facilitate the training for the machine learning classifier.

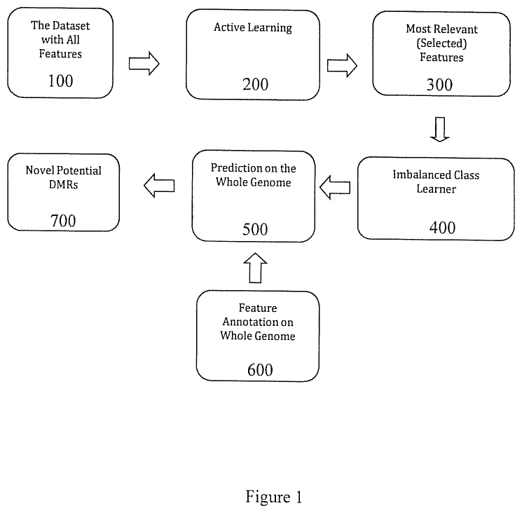

Embodiments of the present invention use a novel two-step (sequential) machine learning analysis involving a combination of an initial active learning step followed by an imbalance class learner (ACL-ICL) protocol (FIG. 1). The computer or server uses a Generalized Query-based Active Learning (GQAL) approach and training sets of data of known epimutations to identify the optimal features associated with the known epimutations. The subsequent ICL takes into consideration the imbalance of the data sets, namely the larger number of non-epimutation sites than epimutation sites in the genome. The computer algorithm then uses a genome wide list of sites with genomic features (a Feature Annotation step) to then predict the potential epimutation sites. This Feature Annotation step involves taking a genome wide list of features and locations on the genome, to then predict the genome wide set of potential epimutations. This technique provides a more tightly integrated approach for a more efficient and accurate machine learning analysis. As shown in the Example presented herein, this novel machine learning technique involves two methods that work synergistically to improve the accuracy and efficiency machine learning and can be used with any type of dataset including biological datasets.

The epigenetics datasets can be from epigenetic transgenerational inheritance experiments and F3 generation sperm or somatic cells from various exposure lineages, including Dioxin [46], Hydrocarbon Jet Fuel [16], Vinclozolin [16,18,19,46], Plastics [15], and Pesticide [12,15]. In some embodiments, somatic Sertoli cells and Granulosa cell datasets [20,21] are derived from adult vinclozolin lineage F3 generation somatic cells that influence the onset of testis and ovarian disease, respectively. The datasets for the germ cell and somatic cell DMR sites [54] have differential DNA methylation changes between the F3 generation exposure and control lineages rat cells. These epigenetic data come from investigations of the actions of environmental exposures during fetal gonadal development that induce epigenetic change in the germ line and promote the epigenetic transgenerational inheritance of adult-onset diseases [3]. The Dioxin, Jet Fuel, Vinclozolin, Plastics and Pesticide datasets consist of ancestral environmental exposures of these five compounds individually and are associated with the epigenetic transgenerational inheritance of adult onset diseases. In some embodiments, the molecular procedure to identify the DMR is a differential methylated DNA immunoprecipitation (MeDIP) followed by a tiling array analysis (Chip) for a MeDIP-Chip analysis. In some embodiments, an additional validation is done using two sperm DMR data sets and a combination of the DDT [14] and MXC [13] sperm epimutations is used as a positive control (DDT MXC with 76 DMR).

In some embodiments, the methods of the invention are used to identify a genome-wide set of potential epimutations that can be used to facilitate identification of epigenetic diagnostics for ancestral environmental exposures and disease susceptibility. As described in the Example herein, the input to the system are datasets with all features. A generalized query based ACL method can be used to find the most important samples and features for the epigenetic datasets. These features are annotated for the epimutation regions, the identified DNA methylation regions (DMRs), as well as sequences upstream and downstream of the DMRs. The most relevant features of each of the datasets are combined and the ACL is trained on these features sets. Once ACL training is complete, ICL training is used for prediction across the whole genome for each germ cell and somatic cell data separately. Once the ICL training is complete, a prediction on the whole genome is made. Thus, the approach allows for the identification of potential new DMRs by first constructing a robust classifier (using the active learning and imbalanced class learning approach) which minimizes false positives, and then scanning the genome for locations which are highly likely to be DMRs. Although previous machine learning approaches applied active learning or imbalance class learning independently, the sequential use for a biological data set is novel.

The methods disclosed herein of using active learning and imbalanced class learning in a combined approach over traditional machine learning classification has distinct advantages. Biological datasets come with a set of inherent problems. Most data that researchers are interested in (e.g. positive cases) are rare (i.e. imbalanced) in contrast to all other characteristics or features. Efficient learning can be performed only when target concepts from both classes (e.g. DMR and non-DMR) are learned well to distinguish them separately while learning from only the relevant features. Such interesting computational problems can be approached using specific machine learning techniques. The present invention allows for the identification of the most relevant features and addresses the class imbalance problem. The genomic characteristics of the DMRs are used as features for the learners. Active learning intelligently chooses the best instances/features to learn from. In some embodiments, the approach uses Generalized Query Based Active Learning (GQAL) which not only can choose the best features to learn from, but also selects the most relevant features for learning. This is accomplished by constructing intelligent queries by removing irrelevant features from the query which an Oracle can answer easily. This approach allows the learner to label multiple instances at the same time instead of labeling one instance per query. In addition, instead of using a global feature reduction (where a set of features are removed in the beginning of the training) GQAL uses a subset of features at each iteration by using local feature selection. This makes use of most of the power of the features and it maximizes the use of a subset of features for learning. The GQAL approach has been tested on 13 datasets besides epigenetics and compared with 3 other classifiers (KNN, SVM and NB) and later with (AdaBoost, Decision Trees, RandomForest and Logistics) and the GQAL was found to be the most efficient for the epigenetic dataset. Aspects of the present invention, combine these two approaches into a single sequential computational tool.

Instead of using an under-sampling or an oversampling technique as done previously to reduce or increase the size of each of the classes to make them balanced, in some embodiments, the approach described herein uses a boosting technique termed AdaBoost or “Adaptive Boosting” [58,59]. Boosting is a method to increase weights of certain examples while decreasing the weights of other examples for efficient balanced learning. This approach allows the learning algorithm to learn target concepts well from both classes. This addresses the imbalance class problem. For the AdaBoost algorithm, a weak classifier termed Tree Augmented Bayesian Network (TAN) [60] may be chosen as the classifier. This is a restrictive Bayesian leamer which performs better than the Naïve Bayes Classifier (NBC) [61]. The TAN boosted imbalances class leamer has been tested on 5 datasets including 2 epigenetic datasets and compared with 2 other imbalanced class learners (Subset Sampling Optimization and EasySensemble) and 5 other regular classifiers (SVM, Logistics, Decision Trees, RandomForest and AdaBoost) [31] and the TAN AdaBoost was found to be the most efficient in the epigenetic dataset.

“Epimutation” and “epigenetic modification” as used herein refer to modifications of cellular DNA that affect gene expression without altering the DNA sequence. The epigenetic modifications are both mitotically and meiotically stable, i.e. after the DNA in a cell (or cells) of an organism has been epigenetically modified, the pattern of modification persists throughout the lifetime of the cell and is passed to progeny cells via both mitosis and meiosis. Therefore, within the organism's lifetime, the pattern of DNA modification and consequences thereof, remain consistent in all cells derived from the parental cell that was originally modified. Further, if the epigenetically modified cell undergoes meiosis to generate gametes (e.g. eggs, sperm), the pattern of epigenetic modification is retained in the gametes and thus inherited by offspring. In other words, the patterns of epigenetic DNA modification are transgenerationally transmissible or inheritable, even though the DNA nucleotide sequence per se has not been altered or mutated. Without being bound by theory, it is believed that enzymes known as methyltransferases shepherd or guide the DNA through the various phases of mitosis or meiosis, reproducing epigenetic modification patterns on new DNA strands as the DNA is replicated.

Exemplary epigenetic modifications include but are not limited to DNA methylation, histone modifications, chromatin structure modifications, and non-coding RNA modifications, etc.

“Epigenetic control region” or “ECR” refers to a segment of DNA which is at least about 400 bp in length, and which is characterized by (contains, comprises, harbors, etc.) at least one of the features described herein, such as differential DNA methylation, a low CpG density (e.g. of about 15% or less), DNA sequence motifs (e.g. EDM1, EDM2), etc. Such DNA segments encompass at least one epimutation and/or at least one epimutation regulatory site. ECRs comprise at least about 400 contiguous base pairs, and may contain up to about 1000 bps (e.g. about 450, 500, 550, 600, 650, 700, 750, 800, 850, 900, 950 or more) base pairs. In some embodiments, the regions are even larger, e.g. about 1000 or more bps. One or more copies of each DNA sequence motif may be present in a region.

Epigenetic modifications may be caused by exposure to any of a variety of factors, examples of which include but are not limited to: chemical compounds e.g. endocrine disruptors such as vinclozolin; chemicals such as those used in the manufacture of plastics e.g. bisphenol A (BPA); bis(2-ethylhexyl)phthalate (DEHP); dibutyl phthalate (DBP); insect repellants such as N, N-diethyl-meta-toluamide (DEET) and dichlorodiphenyltrichloroethane (DDT); pyrethroids such as permethrin; various polychlorinated dibenzodioxins, known as PCDDs or dioxins e.g. 2,3,7,8-tetrachlorodibenzo-p-dioxin (TCDD); hydrocarbon mixtures such as jet fuel; extreme conditions such as abnormal nutrition, starvation, etc.

In some embodiments, the methods as described herein involves obtaining the nucleotide sequence of a selected DNA sequence of interest (e.g. by obtaining a DNA sample from a donor or subject and then sequencing the DNA within the sample; or obtaining a known nucleotide sequence from a database), and then analyzing the nucleotide sequence. Computer executable algorithms and software programs for implementing the same are encompassed by the invention. The software program may contain instructions for causing a computer to carry out the steps of the methods disclosed herein. The computer program will be embedded in a non-transient medium such as a hard drive, DVD, CD, thumb drive, etc.

In some embodiments, the nucleotide sequence of the DNA sequence of interest may be unknown and it may be necessary to carry out a step of sequencing. Those of skill in the art are familiar with techniques that may be used to sequence DNA, including but not limited to: the Maxam-Gilbert chemical degradation method, the Sanger dideoxy chain termination technique, etc. DNA sequencing has been summarized in many review articles, e.g., B. Barrell, The FASEB Journal, 5, 40 (1991); and G. L. Trainor, Anal. Chem. 62, 418 (1990), and references cited therein. The most widely used DNA sequencing chemistry is the enzymatic chain termination method of Sanger, mentioned above, which has been adopted for several different sequencing strategies. The sequencing reactions are either performed in solution with the use of different DNA polymerases, such as the thermophilic Taq DNA polymerase [M. A. Innes, Proc. Natl. Acad. Sci. USA, 85: 9436 (1988)] or specially modified T7 DNA polymerase (“SEQUENASE”) [S. Tabor and C. C. Richardson, Proc. Natl. Acad. Sci. USA, 84,4767 (1987)], or in conjunction with the use of polymer supports. See for example S. Stahl et al., Nucleic Acids Res., 16, 3025 (1988); M. Uhlen, PCT Application WO 89/09282; Cocuzza et al., PCT Application WO 91/11533; and Jones et al., PCT Application WO 92/03575.

In other embodiments, the nucleotide sequences of the DNA sequence(s) of interest have already been determined and are retrieved e.g. from a database. Such databases, many of which are publically available, are well known to those of skill in the art, e.g. GenBank.

Selection of a DNA sequence of interest may be predicated on and/or influenced by any number of factors. For example, the DNA sequence of interest may be from a particular species under study (e.g. a mammalian species, including but not limited to humans); the DNA sequence of interest may be from a particular chromosome or region of a chromosome that is suspected to be involved in a disease or condition of interest; etc. The DNA sequence of interest may be isolated from a subject or subjects known or suspected to be afflicted with a disease or condition associated with epigenetic mutations; or who have been or are suspected of having been exposed to an agent that causes, or is suspected of causing, epigenetic mutations; or who have inexplicably inherited a disease or disease condition from a parent for which no DNA sequence mutation has been identified, etc. Subjects whose DNA is analyzed may be or any age or gender, and in any stage of development, so long as cells containing a DNA sequence of interest can be obtained from the subject. For example, the subject may be an adult, an adolescent, a child, an infant, an embryo, a laboratory animal, etc. The cells from which the DNA is obtained may be any suitable cell, including but not limited to gametes, cells from swabs such as buccal swabs, cells sloughed into amniotic fluid, etc.

The genomic features described herein may be used in a variety of therapeutic applications. For example, they may be used to identify locations of epigenetic modification, or locations that are susceptible to epigenetic modification, within a gene sequence of interest. The gene sequence of interest may be a chromosome or a region of interest within a chromosome. Once identified, such regions can serve as biomarkers to be used e.g. in disease diagnosis and/or to detect environmental exposures to agents or conditions that cause epimutations and/or to monitor therapeutic responsiveness to a medicament or treatment and/or used as prognostic indicators. For example, once a particular location on a chromosome is determined to be a region with a high incidence of epigenetic modifications associated with a particular disease or syndrome, or with exposure to a particular agent or event (e.g. exposure to dioxin), then subjects with or without symptoms of exposure can be screened using a diagnostic that detects epigenetic modification of the region. The detection of epigenetic modification at the region (i.e. a positive diagnostic result) will suggest or confirm that the subject has, indeed, likely been exposed to dioxin, and treatments suitable for dioxin exposure can be instituted. In contrast, a negative result (no epigenetic modification at the site) suggests that the subject has not been exposed to dioxin (or at least that the exposure did not result in damage), and other reasons for disease symptoms displayed by the subject can be investigated. If it is known that exposure did occur, then prophylactic screening of a DNA sample from a patient can result in early identification of a risk of disease and lead to early therapeutic intervention. In addition, ongoing monitoring of the extent of epigenetic modification of a site can provide valuable information regarding the outcome of the administration of agents (e.g. drugs or other therapies) which are intended to treat or prevent a condition caused by epimutation, i.e. the therapeutic responsiveness of a patient. Those of skill in the art will recognize that such analyses are generally carried out by comparing the results obtained using an unknown or experimental sample with results obtained a using suitable negative or positive controls, or both.

Information concerning the type and extent of epigenetic modification in a subject may be used in a variety of decision making processes undertaken by a subject that is tested. For example, depending on the severity of the symptoms caused by an epigenetic modification that is identified, a subject may decide to forego having children or to terminate a pregnancy in order to prevent transmission of the modification to offspring. Diagnostic tests based on the present invention can be included in prenatal testing.

In other embodiments, the regions identified as described herein may be monitored in order to ascertain whether or not administration or exposure to an agent or environmental stimulus causes epimutations. For example, candidate drugs or other treatments that are found to cause epigenetic modifications, for example, in cell or animal studies, or during clinical trials, might be avoided or used only as a last resort in a clinical setting, or rejected altogether as viable drug candidates.

Subjects whose DNA is analyzed may be suffering from any of a variety of disorders (diseases, conditions, etc.) including but not limited to: various known late or adult onset conditions, such as low sperm production, abnormalities of sexual organs, ovarian cysts, kidney abnormalities, prostate disease, immune abnormalities, behavioral effects, etc. In other embodiments, no symptoms are present but screening using the diagnostics is employed to rule out the presence of “silent” epigenetic mutations which could cause disease symptoms in the future, or which could be inherited and cause deleterious effects in offspring.

The regions that are identified as described herein may also be used to screen and identify therapeutic modalities for the treatment of epigenetic mutations. Those of skill in the art will recognize that such methods of screening are typically carried out in vitro, e.g. using a DNA sequence that is immobilized in a vessel, or that is present in a cell. However, such tests may also be carried out in model laboratory animals, once the regions are identified. In one embodiment, candidate agents which reverse epigenetic modification are screened by analyzing the regions. In another embodiment, candidate agents which prevent epigenetic modifications are screened by analyzing the regions. In this way, the epigenetic biomarkers can be used to facilitate, e.g. drug development and clinical trials patient stratification (i.e. pharmacoepigenomics).

The invention also provides a system for carrying out the methods of the invention. The system comprises, for example, i) a computer; and ii) non-transient storage medium comprising computer executable instructions which are performed by the computer and which cause the computer to carry out the steps of a) receiving at least one genomic DNA sequence as input; b) scanning said at least one genomic DNA sequence using Active Learning analysis; and c) scanning said at least one genomic DNA sequence using Imbalance Class Learner analysis wherein said steps b) and c) are performed sequentially or simultaneously. The system also generally comprises iii) an output device capable of presenting results obtained by the computer during or as a result of (e.g. in) scanning steps. The system may further comprise a server wherein said computer executable instructions which are performed by the computer cause the computer to carry out steps b) and c) on the server.

The non-transient storage medium may be on the hard drive of the computer, or may be located on a portable device such as a disc, CD, DVD, thumb drive, flash drive, lap top, portable computer (e.g. a PC or other type), or other such device. Alternatively, the non-transient storage medium may be at a location such as a remote location or a database that is accessible via the internet, or stored in a cloud, or in or on another computer or computer system that is accessible by the computer of the system. The non-transient storage medium may also include instructions for causing the computer to receive, as input, at least one genomic DNA sequence from a nucleotide sequencing apparatus or from a database. The database may be downloaded from a remote site (e.g. via the internet), and/or may be located (stored) on the computer, or may be located on another computer or computer system that is accessible by the computer of the system, or may even be located on a portable device as described above. In other embodiments, the data is downloaded from a gene sequencing apparatus, and the system may also include such an apparatus. If present, the apparatus is operably electronically linked to the computer in a manner that allows data gathered or measured by the sequencing apparatus (e.g. a nucleotide sequence) to be outputted and transmitted to and received as input by the computer.

The computer or server can carry out the analysis of one genomic sequence at a time, or, in some embodiments, can analyze two or more sequences at the same time, e.g. by aligning them and scanning them simultaneously. Similarly, the output device may output the results of the scanning steps for one or multiple sequences at the same time.

The output device may be of any suitable type, including but not limited to a printer, a display (e.g. a monitor that displays the results as a list, as a graph, or in some other suitable format), or a modem that sends out information (e.g. to another output device, to another computer, or to a storage device such as a DVD, CD, etc.).

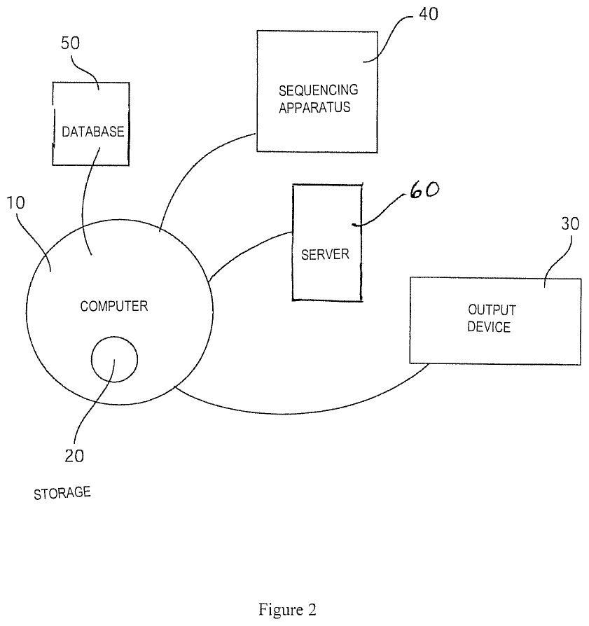

Such a system is illustrated schematically in FIG. 2. FIG. 2 shows computer 10 with non-transient storage medium 20. Computer 10 is operationally linked to (or connected to, functionally connected to, or in electrical communication with) output device 30. In some embodiments, the computer is also operationally linked to nucleic acid sequencing apparatus 40, and data (e.g. a genomic nucleotide sequence, generally a DNA sequence) from nucleic acid sequencing apparatus 40 can be output and transferred to and received as input by computer 10 for analysis by the methods of the invention. In other embodiments, computer 10 is operationally linked to database 50 and information and/or data can be output from database 50 and transferred to and received as input by computer 10. Non-transient storage medium 20 contains computer executable instructions (e.g. code, computer program, etc.) which are performed by the computer and which cause the computer to carry out the steps of the methods described herein. In some embodiments, the computer executable instructions are performed by a server 60 which is operationally linked to the computer 10.

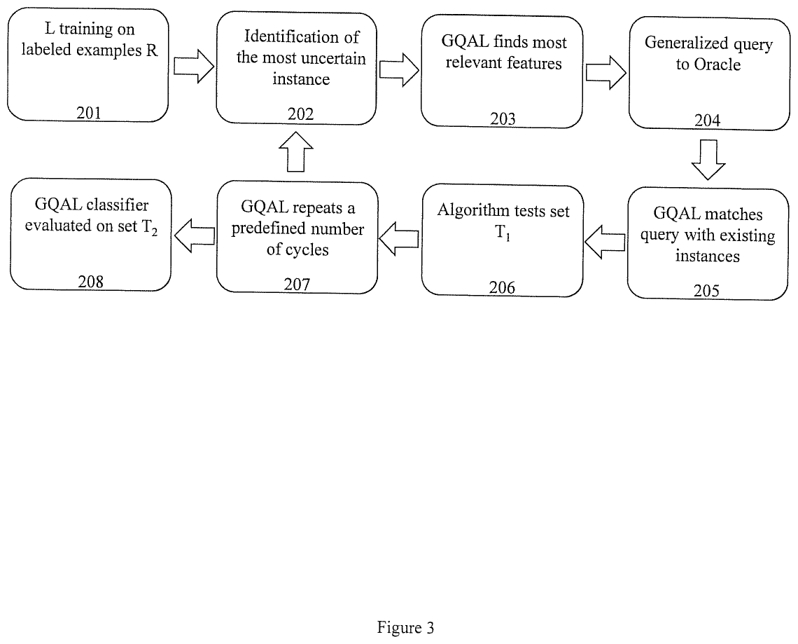

For active learning each of the datasets used can be described as a collection of examples each containing a number of features X₁, X₂ . . . Xₙ and class label Y. Initially the learner is given a small training set R and a set U of unlabeled training instances. From this unlabeled training set, the learner can query the Oracle to label these instances. The Generalized Query Based Active Learning (GQAL) approach is described in the following steps (FIG. 3):

1. Initially, at step 201, the learner L is trained on a small set of labeled examples R, there is a set U of unlabeled training instances, and two separate test sets T₁ and T₂.
2. The classifier learned by leamer L is used on the unlabeled training set U in step 202 to find the most uncertain instance [54].
3. GQAL then takes the chosen uncertain instance and finds the most relevant features for that instance and their ranges in step 203.
4. The process then poses the generalized query in step 204 to the Oracle (Expert), which gives a label and a probability estimation which is the Oracle's confidence about the query label.
5. GQAL takes this generalized query and matches it with existing instances in step 205. Such unlabeled instances are labeled and moved from the unlabeled dataset U to the labeled training set R.
6. The process learns from this updated training set R and tests on the set aside test set T₁ in step 206.
7. GQAL goes back to step 202 and repeats this until it reaches a predefined accuracy or iterates a certain number of times in step 207.
8. Once learning is complete the final GQAL classifier from learner L is evaluated on the set aside test set T₂ in step 208.

In brief, the GQAL takes a large set of features and known training sets having known epimutations, and individually determines the optimal features associated with the known epimutations. This is done for each feature separately and then those features that contribute to the positive identification of the known epimutations during training are selected for future use in the analysis by the Oracle (e.g. human performing the analysis). This is repeated with different training sets and increased number of known epimutations to develop the algorithm used for the subsequent analysis with ICL.

In some embodiments, the Tree Augmented Naive Bayes (TAN) is used as a base classifier for the GQAL learner. Details of this algorithm is given in the GQAL paper [30]. In some embodiments, after running active learning on the entire feature set, the features which appeared as don't care or irrelevant features are removed and features that appeared five times or more are selected as the top features for the dataset. Once the most important features are chosen, they are used for imbalanced class learning which is the next step in the combined approach.

In some embodiments, the ICL uses a boosting technique called AdaBoost that makes use of the entire dataset. It uses a committee of experts (weighted classifiers) to classify any new instance based on majority voting. For the training, initially all instances in the dataset have equal weights. In each iteration AdaBoost increases the weight on the incorrectly classified instances and decreases the weight on the correctly classified instances. After each iteration the classifier, which minimizes the error, is chosen as a committee expert and used to update all the instances for the next iteration. Similar to GQAL the TAN classifier is used as a base classifier with AdaBoost. The objective with the ICL is to correct for imbalance in the data sets. For example, the majority of sites in the genome are non-epimutation sites and a small number are potential epimutations. The ICL corrects for this as described above with established machine learning tools and weighting of the data sets. This contributes to an algorithm that will facilitate the prediction of the potential epimutation sites and genomic locations.

In a combined approach first the active learning is used to select the most important features at each iteration and then the imbalanced class learner is used as a boosting method to maximize the accuracy while learning from an imbalanced dataset. This combined approach (GQAL+(TAN+Adaboost)) is a novel technique than other tightly integrated approaches.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting example which further illustrates the invention, and is not intended, nor should it be interpreted to, limit the scope of the invention.

### Example. Genome-Wide Locations of Potential Epimutations Associated with Environmentally Induced Epigenetic Transgenerational Inheritance of Disease Using a Sequential Machine Learning Prediction Approach

Environmentally induced epigenetic transgenerational inheritance of disease and phenotypic variation involves germline transmitted epimutations. The primary epimutations identified involve altered differential DNA methylation regions (DMRs). Different environmental toxicants have been shown to promote exposure (i.e., toxicant) specific signatures of germline epimutations. Analysis of genomic features associated with these epimutations identified low-density CpG regions (<3 CpG/100 bp) termed CpG deserts and a number of unique DNA sequence motifs. The rat genome was annotated for these and additional relevant features. The objective of the current study was to use a machine learning computational approach to predict all potential epimutations in the genome. A number of previously identified sperm epimutations were used as training sets. A novel machine learning approach using a sequential combination of Active Learning and Imbalance Class Learner analysis was developed. The transgenerational sperm epimutation analysis identified approximately 50K individual sites with a 1 kb mean size and 3,233 regions that had a minimum of three adjacent sites with a mean size of 3.5 kb. A select number of the most relevant genomic features were identified with the low density CpG deserts being a critical genomic feature of the features selected. A similar independent analysis with transgenerational somatic cell epimutation training sets identified a smaller number of 1,503 regions of genome-wide predicted sites and differences in genomic feature contributions. The predicted genome-wide germline (sperm) epimutations were found to be distinct from the predicted somatic cell epimutations. Validation of the genome-wide germline predicted sites used two recently identified transgenerational sperm epimutation signature sets from the pesticides dichlorodiphenyltrichloroethane (DDT) and methoxychlor (MXC) exposure lineage F3 generation. Analysis of this positive validation data set showed a 100% prediction accuracy for all the DDT-MXC sperm epimutations. Observations further elucidate the genomic features associated with transgenerational germline epimutations and identify a genome-wide set of potential epimutations that can be used to facilitate identification of epigenetic diagnostics for ancestral environmental exposures and disease susceptibility.

A previous study used known imprinted genes and associated genomic features in both mouse and humans to predict additional imprinted genes [25,26]. This study identified critical genomic features and demonstrated approximately 600 new potential imprinted genes [25]. Although this previous analysis investigated a distinct epigenetic process (i.e., imprinting), a similar rationale was used in the current study. The approach used known transgenerational sperm epimutation data sets from a variety of exposures as a training set for a machine learning analysis. A similar approach was used with transgenerational somatic cell epimutation data sets to determine differences and similarities between the germline and somatic cell epimutations. The genomic features previously identified and additional features were used to identify genome-wide regions susceptible to become transgenerational epimutations.

The objective is to utilize a novel machine learning approach with known transgenerational sperm epimutations and associated genomic features to predict genome-wide regions that have a susceptibility to develop into transgenerational epimutations. Observations provide insights into the genomic features associated with epimutations and help understand why these sites may be transgenerationally programmed. Previous studies [1,18] have suggested exposure specificity in epimutations, as well as disease susceptibility later in life. Therefore, genome-wide transgenerational epimutation data sets for germ cells and somatic cells will be invaluable in future identification of diagnostics for environmental exposures and later life disease susceptibility.

### Methods

Epigenetic Datasets. The epigenetics datasets are from epigenetic transgenerational inheritance experiments and F3 generation sperm or somatic cells from various exposure lineages, including Dioxin [46], Hydrocarbon Jet Fuel [16], Vinclozolin [16,18,19,46], Plastics [15], and Pesticide [12,15]. The somatic Sertoli cells and Granulosa cell datasets [20,21] are derived from adult vinclozolin lineage F3 generation somatic cells that influence the onset of testis and ovarian disease, respectively. The datasets for the germ cell and somatic cell DMR sites [54] have differential DNA methylation changes between the F3 generation exposure and control lineages rat cells. These epigenetic data come from investigations of the actions of environmental exposures during fetal gonadal development that induce epigenetic change in the germ line and promote the epigenetic transgenerational inheritance of adult-onset diseases [3]. The Dioxin, Jet Fuel, Vinclozolin, Plastics and Pesticide datasets consist of ancestral environmental exposures of these five compounds individually and are associated with the epigenetic transgenerational inheritance of adult onset diseases. The molecular procedure to identify the DMR was a differential methylated DNA immunoprecipitation (MeDIP) followed by a tiling array analysis (Chip) for a MeDIP-Chip analysis and the details of how each experiment was performed and data was collected is previously described [18,20,21]. An additional validation was done using two recently identified sperm DMR data sets. A combination of the DDT [14] and MXC [13] sperm epimutations is used as a positive control (DDT MXC with 76 DMR).

Active Learning. For active learning each of the datasets used can be described as a collection of examples each containing a number of features X₁, X₂ . . . Xₙ and class label Y. Initially the learner is given a small training set R and a set U of unlabeled training instances. From this unlabeled training set, the learner can query the Oracle to label these instances. The GQAL approach is described in the following steps:

1. Initially the learner L is trained on a small set of labeled examples R, there is a set U of unlabeled training instances, and two separate test sets T₁ and T₂.
2. The classifier learned by learner L is used on the unlabeled training set U to find the most uncertain instance [54].
3. GQAL then takes the chosen uncertain instance and finds the most relevant features for that instance and their ranges.
4. The algorithm poses the generalized query to the Oracle, which gives a label and a probability estimation which is the Oracle's confidence about the query label.
5. GQAL will take this generalized query and match it with existing instances. Such unlabeled instances are labeled and moved from the unlabeled dataset U to the labeled training set R.
6. The algorithm learns from this updated training set R and tests on the set aside test set T₁.
7. GQAL goes back to step 2 and repeats this until it reaches a predefined accuracy or iterates a certain number of times.
8. Once learning is complete the final GQAL classifier from learner L is evaluated on the set aside test set T₂.

The Tree Augmented Naive Bayes (TAN) is used as a base classifier for the GQAL learner. Details of this algorithm is given in the GQAL paper [30]. After running active learning on the entire feature set of 834 features, the features which appeared as don't care or irrelevant features were removed and features that appeared five times or more were selected as the top features for the dataset. This ended up being 149 features for SG and 134 features for DHVPP. The entire list of genomic features is given in Tables 1 and 2. They are grouped into CpG information, repeat elements, transcription factors, sequence motifs and mammalian motifs. Once the most important features were chosen, they were used for imbalanced class learning which is the next step in the combined approach.

TABLE 1
ACL selected features in the germ cell DHVPP (Dioxin, Hydrocarbon
(Jet Fuel), Vinclozolin, Plastics, Pesticide) final feature list (134).
Up denotes upstream, Dn denotes downstream, features without Up
and Dn initial have been extracted from the base region itself.

| CpG | Repeat | | Transcription | | Sequence | | Mammalian | |
|---|---|---|---|---|---|---|---|---|
| Element | Elements | Location | Factors | Location | Motifs | Location | Motifs | Location |
| CpG | A.elements | 5kUp, | Octob1 | 100kDn | CACGTG | DMR, | MCS10.2 | 100kDn |
| density | | 5kDn, | | | | 100kDn | | |
| | | 100kUp | | | | | | |
| | A.elements. | 1kUp | ATF.AC0.2 | DMR | CCGG | DMR, | MCS10.2.2 | 100kUp |
| | count | | | | | 100kDn | | |
| | Alu.B1 | 100kUp | ATSequence | DMR | EDM2B1 | 100kUp, | MCS10.2.3 | 100kUp |
| | | | | | | 100kDn | | |
| | Alu.B1. | 5kDn | ATTTTTTTAT | 100kUp | GCGC | DMR, | MCS10.2.4 | 100kUp |
| | count | | TTTTATTTTA | | | 100kDn | | |
| | | | TTTTTTTTTT | | | | | |
| | | | TTAAAA | | | | | |
| | DNA. | 100kDn | CCGC + ACOA | DMR | TCGG | DMR, | MCS10.3.1 | 100kDn |
| | elements. | | [GT]G- | | | 100kUp | | |
| | count | | GG + ACO- | | | | | |
| | | | GGC | | | | | |
| | ERV_class | 1kUp | CTCF.ACO. | DMR | TGGAGG | DMR | MCS10.4.2 | 100kUp |
| | I | | binding | | GGCAGT | | | |
| | | | | | CCGGCT | | | |
| | | | | | CCTGGG | | | |
| | | | | | GG | | | |
| | ERV_class | 1kUp, | Ddit3 . . . | DMR | | | MCS10.7 | 100kUp |
| | I.count | 100kUp | Cebpa | | | | | |
| | ERV_class | 100kDn | Down | 100kDn | | | MCS11.0.3 | 100kDn |
| | II.count | | Methylation | | | | | |
| | ERVL. | 1kUp, | E2F1 | DMR. | | | MCS11.2.2 | 100kUp |
| | count | 100kUp, | | 100kDn | | | | |
| | | 100kDn | | | | | | |
| | ERVL. | 100kUp | EDM2B1 | DMR | | | MCS11.3.1 | 100kUp |
| | MaLRs | | | | | | | |
| | HAT. | 5kDn | Foxd3 | DMR | | | MCS11.4 | 100kUp |
| | Charlie | | | | | | | |
| | HAT. | 5kUp | FOXP1 | DMR | | | MCS11.5 | 100kUp, |
| | Charlie. | | | | | | | 100kDn |
| | count | | | | | | | |
| | L3.CR1 | 1kUp | HIF1A . . . | DMR | | | MCS11.5.1 | 100kDn |
| | | | ARNT.AKA. | | | | | |
| | LINE2. | 100kUp | InsutorProtein | DMR | | | MCS11.7 | 100kUp |
| | count | | | | | | | |
| | LTR. | 1kUp, | KROX | DMR | | | MCS11.9 | 100kDn |
| | elements | 1kDn | | | | | | |
| | LTR. | 100kDn | MAZ | DMR, | | | MCS12.2 | 100kUp |
| | elements. | | | 100kUp | | | | |
| | count | | | | | | | |
| | MIRs | 5kDn | Nrf2. | DMR | | | MCS12.2.9 | 100kDn |
| | | | GABPA | | | | | |
| | MIRs. | 1kUp, | SOX10 | DMR, | | | MCS12.3.9 | 100kUp |
| | count | 100kDn | | 100kDn | | | | |
| | Simple | 1kDn | Sp1 | DMR | | | MCS13.0 | 100kDn |
| | SINEs | 1kDn | TCTCTGCAG | DMR | | | MCS13.2.1 | 100kUp |
| | SrNEs. | 5kUp, | TGTCTGCAG | DMR | | | MCS13.7.9 | 100kDn |
| | count | 1kDn | | | | | | |
| | Total. | 5kUp, | TGTTTGCAG | 100kUp | | | MCS13.9 | 100kUp |
| | interspersed. | 5kDn, | | | | | | |
| | repeats | 1kDn | | | | | | |
| | | | Zfp423.AKA. | 100kDn | | | MCS14.0 | 100kUp |
| | | | ZNF219 | 100kDn | | | MCS14.3.2 | 100kUp |
| | | | | | | | MCS15.8 | 100kDn |
| | | | | | | | MCS16.1 | 100kDn |
| | | | | | | | MCS16.2 | 100kUp |
| | | | | | | | MCS17.1.1 | 100kUp |
| | | | | | | | MCS17.2 | 100kDn |
| | | | | | | | MCS17.3 | 100kDn |
| | | | | | | | MCS18.8 | 100kDn |
| | | | | | | | MCS21.1.1 | 100kUp |
| | | | | | | | MCS22.1 | 100kDn |
| | | | | | | | MCS22.5 | 100kUp, |
| | | | | | | | | 100kDn |
| | | | | | | | MCS22.8 | 100kDn |
| | | | | | | | MCS23.8 | 100kUp, |
| | | | | | | | | 100kDn |
| | | | | | | | MCS24.3.1 | 100kUp |
| | | | | | | | MCS25.2 | 100kDn |
| | | | | | | | MCS27.2 | 100kDn |
| | | | | | | | MCS30.5 | 100kDn |
| | | | | | | | MCS32.3.1 | 100kDn |
| | | | | | | | MCS37.4 | 100kDn |
| | | | | | | | MCS43.9 | 100kDn |
| | | | | | | | MCS47.6 | 100kDn |
| | | | | | | | MCS69.5 | 100kDn |
| | | | | | | | MCS8.1 | 100kDn |
| | | | | | | | MCS9.0 | 100kUp |
| | | | | | | | MCS9.5 | 100kUp |
| | | | | | | | MCS9.5.1 | 100kUp, |
| | | | | | | | | 100kDn |
| | | | | | | | MCS9.6 | 100kUp |
| | | | | | | | MCS9.6.1 | 100kUp, |
| | | | | | | | | 100kDn |
| | | | | | | | MCS9.8 | 100kUp |
| | | | | | | | MCS9.8.4 | 100kDn |

TABLE 2
ACL selected features in the somatic cell (SG) (Sertoli-Granulosa) final
feature list (149). Up denotes upstream, Dn denotes downstream, features
without Up and Dn initial have been extracted from the base region itself.

| CpG | Repeat | | Transcription | | Sequence | | Mammalian | |
|---|---|---|---|---|---|---|---|---|
| Element | Elements | Location | Factors | Location | Motifs | Location | Motifs | Location |
| CpG | A. | 100kDn | AP2 | 100kUp | CACGTG | DMR, | MCS10.2 | 100kDn |
| density | elements | | | | | 100kDn | | |
| | A. | 100kDn | ATF.AC0.2 | 100kUp | TCGG | 100kUp, | MCS10.2.1 | 100kDn |
| | elements. | | | | | 100kDn | | |
| | count | | | | | | | |
| | Alu.B1 | 5kDn | ATSequence | 100kDn | | | MCS10.22 | 100kDn |
| | Alu.B1. | 5kUp, | AZF1 | 100kUp, | | | MCS10.2.3 | 100kUp, |
| | count | 5kDn, | | 100kDn | | | | 100kDn |
| | | 100kUp | | | | | | |
| | B2.B4 | 5kDn, | CHR | DMR, | | | MCS10.2.4 | 100kDn |
| | | 100kUp | | 100kDn | | | | |
| | B2.B4. | 100kUp | CREB1 | DMR, | | | MCS10.3 | 100kDn |
| | count | | | 100kUp, | | | | |
| | | | | 100kDn | | | | |
| | ERVL. | 100kUp | CTCF.ACO. | 100kUp, | | | MCS10.3.1 | 100kDn |
| | count | | binding | 100kDn | | | | |
| | HAT. | 100kUp | DR.AC0.2 | 100kDn | | | MCS10.7 | 100kUp |
| | Charlie | | | | | | | |
| | HAT. | 100kUp, | E2F1 | 100kUp, | | | MCS10.9 | 100kUp |
| | Charlie. | 100kDn | | 100kDn | | | | |
| | count | | | | | | | |
| | IDS | 100kDn | GC | DMR | | | MCS107.8 | 100kUp |
| | IDS. | 100kDn | HIF1A..ARNT. | 100kDn | | | MCS11.1 | 100kUp |
| | count | | AKA. | | | | | |
| | LINE1 | 1kUp, | KBS | 100kUp | | | MCSU.1.1 | 100kUp |
| | | 5kDn | | | | | | |
| | LINE1. | 1kUp | KROX | DMR, | | | MCS11.1.3 | 100kUp |
| | count | | | 100kUp | | | | |
| | LINE2 | 100kDn | Mafb | DMR | | | MCS11.3 | 100kDn |
| | LINEs | 100kUp, | MAZ | DMR | | | MCS11.3.1 | 100kUp, |
| | | 100kDn | | | | | | 100kDn |
| | LTR. | 100kUp, | Methylation | DMR, | | | MCS12.0 | 100kDn |
| | elements. | 100kDn | | 100kUp | | | | |
| | count | | | | | | | |
| | MIRs | 100kUp | Methylation | 100kDn | | | MCS12.1 | 100kUp |
| | MIRs. | 5kUp | Methylation | 100kDn | | | MCS12.2 | 100kUp |
| | count | | | | | | | |
| | Simple | 100kUp | NFATC2 | 100kUp, | | | MCS12.2.1 | 100kUp |
| | | | | 100kDn | | | | |
| | SINEs | 5kUp | NFYA | DMR | | | MCS12.2 | 100kUp |
| | SINEs. | 5kUp, | Nrf2.GABPA | DMR, | | | MCS12.7 | 100kDn |
| | count | 5kDn, | | 100kDn | | | | |
| | | 100kDn | | | | | | |
| | Total, | 1kUp, | SOX10 | 100kDn | | | MCS12.7.1 | 100kDn |
| | interspersed. | 5kDn | | | | | | |
| | repeats | | | | | | | |
| | A. | 100kDn | Sp1 | 100kUp, | | | MCS13.2.1 | 100kUp |
| | elements | | | 100kDn | | | | |
| | | | Sp1.1 | DMR, | | | MCS13.9 | 100kUp |
| | | | | 100kUp | | | | |
| | | | TGTCTGCAG | 100kUp, | | | MCS14.1 | 100kDn |
| | | | | 100kDn | | | | |
| | | | USF1. | 100kUp, | | | MCS14.3 | 100kDn |
| | | | AC0. | 100kDn | | | | |
| | | | binding | | | | | |
| | | | ZBTB4. | 100kDn | | | MCS14.3 | 100kDn |
| | | | AC0. | | | | | |
| | | | binding | | | | | |
| | | | | | | | MCS14.9 | 100kDn |
| | | | | | | | MCS14.9.1 | 100kDn |
| | | | | | | | MCS15.0 | 100kUp |
| | | | | | | | MCS16.1 | 100kUp |
| | | | | | | | MCS17.2 | 100kDn |
| | | | | | | | MCS17.4 | 100kUp, |
| | | | | | | | | 100kDn |
| | | | | | | | MCS19.1 | 100kUp |
| | | | | | | | MCS19.1.1 | 100kUp |
| | | | | | | | MCS19.8 | 100kDn |
| | | | | | | | MCS21.6 | 100kDn |
| | | | | | | | MCS22.1 | 100kUp |
| | | | | | | | MCS22.5 | 100kUp |
| | | | | | | | MCS23.4 | 100kUp, |
| | | | | | | | | 100kDn |
| | | | | | | | MCS23.8 | 100kUp |
| | | | | | | | MCS24.3.1 | 100kUp |
| | | | | | | | MCS25.2 | 100kUp |
| | | | | | | | MCS25.7 | 100kDn |
| | | | | | | | MCS26.4 | 100kUp |
| | | | | | | | MCS30.0 | 100kDn |
| | | | | | | | MCS30.5 | 100kUp, |
| | | | | | | | | 100kDn |
| | | | | | | | MCS30.8 | 100kDn |
| | | | | | | | MCS32.3.1 | 100kUp |
| | | | | | | | MCS33.5 | 100kDn |
| | | | | | | | MCS37.3 | 100kDn |
| | | | | | | | MCS37.4 | 100kUp |
| | | | | | | | MCS40.4 | 100kDn |
| | | | | | | | MCS43.9 | 100kDn |
| | | | | | | | MCS44.8 | 100kUp |
| | | | | | | | MCS46.0 | 100kUp |
| | | | | | | | MCS47.6 | 100kUp |
| | | | | | | | MCS51.6 | 100kUp |
| | | | | | | | MCS64.6 | 100kUp |
| | | | | | | | MCS8.1 | 100kUp |
| | | | | | | | MCS80.4 | 100kUp |
| | | | | | | | MCS9.1 | 100kDn |
| | | | | | | | MCS9.1.1 | 100kUp, |
| | | | | | | | | 100kDn |
| | | | | | | | MCS9.8 | 100kDn |
| | | | | | | | MCS9.8.1 | 100kUp |

Imbalanced Class Learner. The ICL uses a boosting technique called AdaBoost that makes use of the entire dataset. It uses a committee of experts (weighted classifiers) to classify any new instance based on majority voting. For the training, initially all instances in the dataset have equal weights. In each iteration AdaBoost increases the weight on the incorrectly classified instances and decreases the weight on the correctly classified instances. After each iteration the classifier which minimizes the error is chosen as a committee expert and used to update all the instances for the next iteration. Similar to GQAL the TAN classifier is used as a base classifier with AdaBoost.

The two-step DMR identification machine learning framework is as shown in FIG. 1, starting from the “Dataset” component. Details of each method are presented in earlier reports [30,31]. In a combined approach first the active learning is used to select the most important features at each iteration and then the imbalanced class learner is used as a boosting method to maximize the accuracy while learning from an imbalanced dataset. This combined approach (GQAL+(TAN+Adaboost)) is a newer technique than other tightly integrated approaches.

Both the GQAL and TAN+AdaBoost approach were trained with 10 fold cross validation with the DHVPP and SG data. The models created from these two training sets were separately tested for validity using the MXC-DDT and Sox9SryTcf21 datasets. Validation results show that both the datasets SG and DHVPP can identify DMR dataset MXC-DDT properly and can identify non-DMR, non-epigenetic dataset Sox9SryTcf21 as non-DMR with some restrictions.

Clustering. After the potential DMR sites (1,503 for SG and 3,233 for DHVPP) were extracted, further analysis of the data was done to find if these novel potential DMR sites cluster in certain locations in the genome. A previous study with tissue gene expression array data was used in a cluster analysis of transgenerational differentially expressed genes to identify gene clusters with statistically significant over-represented gene expression [35]. These locations were termed Epigenetic Control Regions (ECRs). A similar analysis for DMR sites was done to find whether such ECR regions exist for the predicted epimutation sites. An overlapping sliding window size of 2,000,000 base was used at an interval of 50,000 base to count the number of potential DMR within the sliding windows. Then a Z-test was performed and p-value of 0.05 statistically significant cut-off, including false discovery analysis, was used to find the windows with over-representations of predicted DMR sites. Then consecutive overlapping windows were merged to form the final list of clusters.

Feature Extraction. The feature extraction included using RepeatMasker, Motif discovery tools and consensus sequences obtained from JASPER and other sources [20]. Features were extracted from the base region, 1 k, 5 k and 100 k upstream and downstream. A non-overlapping region of 1000 bases was used to scan all the chromosomes of the rat to create the testing regions and then features were collected from these regions and around it (having the 1000 bases as a base region). The same features were used for training and testing for each individual dataset.

### Results

The machine learning approach used in this study (FIG. 1) uses the generalized query based ACL method to find the most important samples and features for the epigenetic datasets. Initially the number of features collected for the epigenetic dataset was 834 for each of the two transgenerational datasets. The germ cell (sperm) dataset was Dioxin-Hydrocarbons (Jet Fuel)-Vinclozolin-Plastics-Pesticides (DHVPP), and somatic cell dataset was Sertoli-Granulosa (SG). Table 3 contains descriptions of the different epimutation datasets.

TABLE 3
Description of epimutation datasets: germ cell DHVPP;
somatic cell (SG); MXC-DDT; and non-DMR Sox9SryTcf21.

| DataSet Name | Description |
|---|---|
| Germ Cell | Ancestral environmental exposures (Dioxin, |
| (DHVPP) | Hydrocarbon Jet Fuel, Vinclozolin, Plastics, |
| | Pesticide) transgenerational germ cell |
| | epimutations. |
| Somatic Cell | Adult somatic cell (Sertoli and Granulosa |
| (SG) | cell) transgenerational epimutations from |
| | F3 generation vinclozolin rats. |
| Validation Set | Pesticide Methoxychlor and Dichlorodiphenyl- |
| (MXCDDT) | trichloroethane (DDT) exposures promote the |
| | epigenetic transgenerational inheritance of |
| | germ cell epimutations the F3 generation rats. |
| Negative Set | Testicular Sertoli cell differentiation |
| (Sox9SryTcf21) | transcription factor Sox9, Sry and Tcf21 |
| | binding sites (non-DMR). |

The selected 834 genomic features can be grouped into four sub-groups (Table 4). They are CpG density and related information (3 total features), repeat elements (216 total features), transcription factors (207 total features) and DNA sequence motifs (60 total features). The sequence motif group has a subgroup called mammalian motifs (348 total features) as these features were collected from the online JASPER dataset [32]. All these features were annotated for the epimutation regions (the identified DMR regions), as well as for sequences 1 k, 5 k, and 100 k upstream and downstream of the DMRs. ACL was run on the DHVPP and SG datasets separately and only those features that appeared greater than 5 times, as well as some manually selected important features were chosen as the most relevant features for further analysis (Tables 5 and 6). This information for each of these datasets was combined and ACL trained on these feature sets. Once ACL training was complete ICL training was used for prediction across the whole genome for each germ cell and somatic cell data set separately (FIG. 1).

TABLE 4
Initial set of features for DMR identification (834). Up/down
indicates features collected from upstream and downstream.

| | CpG, | | | | |
|---|---|---|---|---|---|
| Feature | length, | | Tran- | | |
| Location | CpG | Repeat | scription | Sequence | Mammalian |
| & Number | density | Element | Factors | Motifs | Motifs |
| DMR (92) | 3 | | 69 | 20 | |
| 1k up/down | | 72 | | | |
| stream (72) | | | | | |
| 5k up/down | | 72 | | | |
| stream (72) | | | | | |
| 100k up/down | | 72 | 138 | 40 | 348 |
| stream (598) | | | | | |
| Total features | 3 | 216 | 207 | 60 | 348 |
| (834) | | | | | |

TABLE 5
Final distribution of selected features (134) for
the germ cell DHVPP dataset. ACL selected deatures.
Most prominent features (for combined datasets).

| | CpG, | | | | |
|---|---|---|---|---|---|
| Feature | length, | | Tran- | | |
| Location | CpG | Repeat | scription | Sequence | Mammalian |
| & Number | density | Element | Factors | Motifs | Motifs |
| Base region | 1 | | 23 | 5 | |
| 1k up/down | | 12 | | | |
| stream | | | | | |
| 5k up/down | | 9 | | | |
| stream | | | | | |
| 100k up/down | | 11 | 9 | 6 | 58 |
| stream | | | | | |

TABLE 6
Final distribution of selected features (149) for
the somatic cell SG dataset. ACL selected deatures.
Most prominent features (for combined datasets).

| | CpG, | | | | |
|---|---|---|---|---|---|
| Feature | length, | | Tran- | | |
| Location | CpG | Repeat | scription | Sequence | Mammalian |
| & Number | density | Element | Factors | Motifs | Motifs |
| Base region | 1 | | 10 | 1 | |
| 1k up/down | | 3 | | | |
| stream | | | | | |
| 5k up/down | | 10 | | | |
| stream | | | | | |
| 100k up/down | | 19 | 31 | 3 | 71 |
| stream | | | | | |

Since most of the DMR locations are found within 600 bp to 1500 bp windows, a non-overlapping sliding window of 1000 bp was used on each chromosome to identify potential DMR candidate sites. The original 834 selected genomic features were extracted/annotated for the entire rat genome DNA sequence. The number of initial extracted/annotated feature sets is shown in Table 4. For each of the 21 rat chromosomes (autosomes and X chromosome) a sliding non-overlapping window size of 1000 bases was used to create a total of 2,630,424 sites. In the same manner as the training dataset, FASTA files were created. RepeatMasker was run and finally a list of 834 features was extracted from each of these sites. This is the test set used for prediction. Once the training was complete, a prediction on the whole genome was made. This approach to find potential new DMRs is the first to construct a robust classifier (using both imbalanced class and active learning approach) which minimizes false positives, and then scan the genome for locations which are highly likely to be DMRs, FIG. 1.

Once these features were identified, annotated and extracted from the training datasets, active learning was used to find the most relevant features. The features which appeared 5 or less times were considered don't care attributes (irrelevant features) and a set of manually selected features was taken as the list of most relevant features. The most relevant features for the two training datasets are presented in Tables 1, 5, and 6. The list of features include the following categories: (a) CpG information (b) repeat elements (c) transcription factors (d) sequence motifs and (e) mammalian motifs. The CpG Information contains three features: length of the sites in base pair, number of CpG sites, and CpG density (number of CpG sites per 100 bases). The transgenerational epimutations have been found in low CpG density regions (termed CpG deserts) [22]. The genomic feature of low CpG density was found to be one of the most important features for both the somatic and germ cell prediction datasets. The repeat elements original list contained a total of 216 repeat features. Both the somatic and sperm datasets had 32 repeat elements (with significant overlaps) in their final list of somatic 134 and sperm 149 features (Tables 4-6). The original transcription factor group contained 207 features. In the final list for sperm (DHVPP) there were 32 transcription factor features and for the somatic cells (SG) there were 41 features. The DNA sequence motifs [33,34] had 60 original features selected for this study. For the sperm (DHVPP) dataset there are 11 sequence motif features and for the somatic (SG) dataset there are 4 sequence motifs critical features. Mammalian motifs originally considered involved 348 features from the JASPER dataset [32]. For the sperm (DHVPP) there were 58 mammalian motif features while for the somatic cell (SG) there were 71 of them (Tables 5 and 6).

Once the final list of features was selected for the two datasets they were used for training in the ICL, and used for the genome wide prediction. The sperm and somatic cell analysis was done separately with the relevant list for each. The initial number of predicted epimutation sites identified was 48,557 sites for the sperm (DHVPP) and 28,564 sites for the somatic cells (SG). However, after an initial number of individual sites were found, only those with three or more consecutive sites were merged to create the most stringent list of potential susceptible DMR sites. The reason for focusing on three or more consecutive sites is that single predicted sites have a lower statistical significance and a higher potential for false positives. Although the single sites are viable potential DMR to consider, a more stringent analysis of DMR was used of three or more consecutive probes being present to further investigate the potential differential DNA methylation regions. These three or more consecutive sites were merged to create the list of potential susceptible DMR sites. The final list of potential DMR for the sperm DHVPP analysis was 3,233 sites and for the somatic cell SG analysis was 1,503 sites.

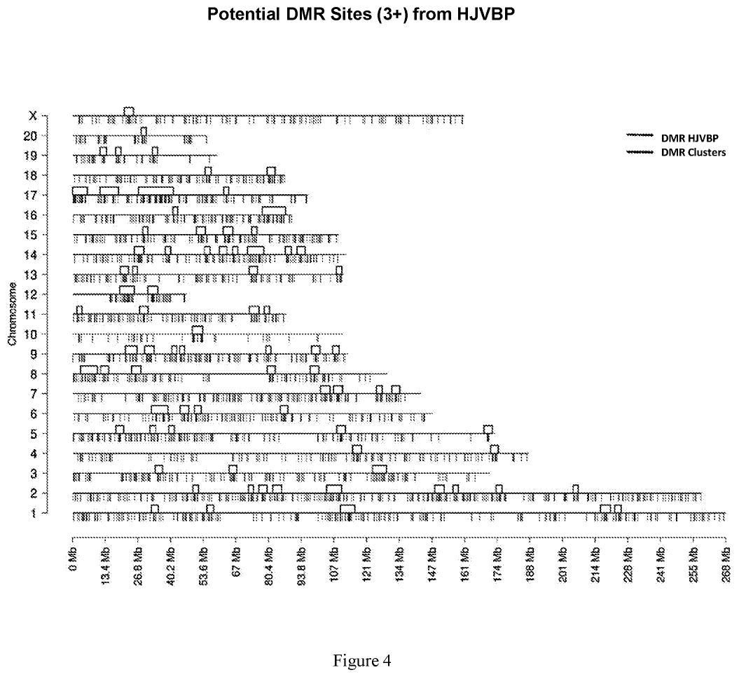

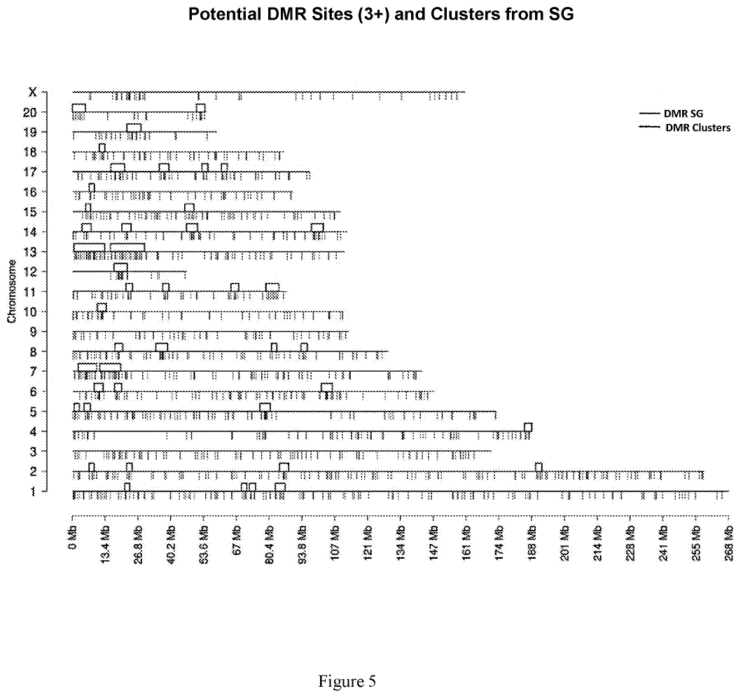

The chromosome plots for the datasets DHVPP (FIG. 4) and SG (FIG. 5) are presented and the predicted DMR/epimutation regions are shown on all chromosomes. Once the three or more consecutive sites were identified a cluster analysis was performed to identify DMR co-localization. The methods section describes the cluster construction procedure for the identification of statistically significant over-represented within the regions DMR. A total of 80 clusters were formed from the predicted 3,233 DMR sites for the germline (DHVPP) dataset as shown in FIG. 4. The average size of the germline DMR clusters were 3,574,375 bases and 32% of the total sites fall within those clusters. For the somatic cell (SG) dataset a total of 44 DMR clusters were identified from the predicted 1508 DMR sites. Average cluster sizes are 4,046,591 bases long and 27% of the total sites fall within these clusters, FIG. 5. The list of predicted cluster regions is presented in Tables 7 and 8 and shown in FIGS. 4 and 5. These DMR clusters demonstrate that the potential DMRs are in part localized in certain regions of the genome. These clusters of potential DMRs are speculated to act as Epigenetic Control Regions (ECR) to regulate gene expression within the clusters [35].

TABLE 7
Clusters from combined datasets and stats (cluster size,
number of sites in each cluster). Clusters from predicted
germ cell DMRs (from 3+ consecutive sites only) (80).

| | Chromosome | cSTART | cSTOP | Length |
|---|---|---|---|---|
| 1 | chr1 | 32350000 | 35200000 | 2850000 |
| 2 | chr1 | 55100000 | 57850000 | 2750000 |
| 3 | chr1 | 109900000 | 115750000 | 5850000 |
| 4 | chr1 | 216550000 | 220500000 | 3950000 |
| 5 | chr1 | 222350000 | 224900000 | 2550000 |
| 6 | chr10 | 49100000 | 53400000 | 4300000 |
| 7 | chr11 | 1850000 | 3900000 | 2050000 |
| 8 | chr11 | 27550000 | 30950000 | 3400000 |
| 9 | chr11 | 72550000 | 76500000 | 3950000 |
| 10 | chr11 | 78600000 | 80750000 | 2150000 |
| 11 | chr12 | 19300000 | 25350000 | 6050000 |
| 12 | chr12 | 30900000 | 34950000 | 4050000 |
| 13 | chr13 | 19600000 | 22900000 | 3300000 |
| 14 | chr13 | 24600000 | 26600000 | 2000000 |
| 15 | chr13 | 72500000 | 75800000 | 3300000 |
| 16 | chr13 | 108200000 | 110450000 | 2250000 |
| 17 | chr14 | 25300000 | 29250000 | 3950000 |
| 18 | chr14 | 3.80E+007 | 40200000 | 2200000 |
| 19 | chr14 | 54100000 | 56400000 | 2300000 |
| 20 | chr14 | 60150000 | 63350000 | 3200000 |
| 21 | chr14 | 65650000 | 67700000 | 2050000 |
| 22 | chr14 | 71800000 | 78400000 | 6600000 |
| 23 | chr14 | 87300000 | 89750000 | 2450000 |
| 24 | chr14 | 92050000 | 95350000 | 3300000 |
| 25 | chr15 | 28900000 | 30900000 | 2000000 |
| 26 | chr15 | 50800000 | 54550000 | 3750000 |
| 27 | chr15 | 61800000 | 65750000 | 3950000 |
| 28 | chr15 | 73450000 | 75650000 | 2200000 |
| 29 | chr16 | 41200000 | 43200000 | 2000000 |
| 30 | chr16 | 77850000 | 87400000 | 9550000 |
| 31 | chr17 | 50000 | 6050000 | 6000000 |
| 32 | chr17 | 11200000 | 18850000 | 7650000 |
| 33 | chr17 | 27100000 | 41300000 | 14200000 |
| 34 | chr17 | 6.20E+007 | 6.40E+007 | 2000000 |
| 35 | chr18 | 54350000 | 56850000 | 2500000 |
| 36 | chr18 | 79850000 | 83100000 | 3250000 |
| 37 | chr19 | 11250000 | 1.40E+007 | 2750000 |
| 38 | chr19 | 17650000 | 19850000 | 2200000 |
| 39 | chr19 | 32750000 | 34850000 | 2100000 |
| 40 | chr2 | 49350000 | 51700000 | 2350000 |
| 41 | chr2 | 72050000 | 74200000 | 2150000 |
| 42 | chr2 | 76400000 | 79800000 | 3400000 |
| 43 | chr2 | 82100000 | 85800000 | 3700000 |
| 44 | chr2 | 104200000 | 110300000 | 6100000 |
| 45 | chr2 | 148450000 | 152400000 | 3950000 |
| 46 | chr2 | 1.56E+008 | 158200000 | 2200000 |
| 47 | chr2 | 173650000 | 176100000 | 2450000 |
| 48 | chr2 | 205300000 | 207400000 | 2100000 |
| 49 | chr20 | 28250000 | 30250000 | 2000000 |
| 50 | chr3 | 33850000 | 36900000 | 3050000 |
| 51 | chr3 | 64150000 | 67400000 | 3250000 |
| 52 | chr3 | 1.23E+008 | 128700000 | 5700000 |
| 53 | chr4 | 114900000 | 118550000 | 3650000 |
| 54 | chr4 | 171450000 | 174550000 | 3100000 |
| 55 | chr5 | 17800000 | 2.10E+007 | 3200000 |
| 56 | chr5 | 31800000 | 34150000 | 2350000 |
| 57 | chr5 | 39500000 | 41800000 | 2300000 |
| 58 | chr5 | 108350000 | 111800000 | 3450000 |
| 59 | chr5 | 168750000 | 172100000 | 3350000 |
| 60 | chr6 | 32400000 | 39150000 | 6750000 |
| 61 | chr6 | 44100000 | 47700000 | 3600000 |
| 62 | chr6 | 49900000 | 52750000 | 2850000 |
| 63 | chr6 | 85250000 | 88250000 | 3000000 |
| 64 | chr7 | 101650000 | 105600000 | 3950000 |
| 65 | chr7 | 1.07E+008 | 110700000 | 3700000 |
| 66 | chr7 | 124550000 | 1.27E+008 | 2450000 |
| 67 | chr7 | 1.31E+008 | 134200000 | 3200000 |
| 68 | chr8 | 3300000 | 10150000 | 6850000 |
| 69 | chr8 | 11600000 | 14850000 | 3250000 |
| 70 | chr8 | 24200000 | 28050000 | 3850000 |
| 71 | chr8 | 79900000 | 83150000 | 3250000 |
| 72 | chr8 | 97400000 | 1.01E+008 | 3600000 |
| 73 | chr9 | 21700000 | 26500000 | 4800000 |
| 74 | chr9 | 29550000 | 33500000 | 3950000 |
| 75 | chr9 | 40650000 | 42750000 | 2100000 |
| 76 | chr9 | 43950000 | 46050000 | 2100000 |
| 77 | chr9 | 79300000 | 81350000 | 2050000 |
| 78 | chr9 | 9.80E+007 | 101500000 | 3500000 |
| 79 | chr9 | 106650000 | 109350000 | 2700000 |
| 80 | chrX | 21250000 | 2.50E+007 | 3750000 |

TABLE 8
Clusters from combined datasets and stats (cluster size, number
of sites in each cluster). Clusters from Sertoli-Granulosa
predicted DMRs (from 3+ consecutive sites only) (44).

| | Chromosome | cSTART | cSTOP | Length |
|---|---|---|---|---|
| 1 | chr1 | 21450000 | 23450000 | 2000000 |
| 2 | chr1 | 6.90E+007 | 71300000 | 2300000 |
| 3 | chr1 | 72400000 | 74700000 | 2300000 |
| 4 | chr1 | 82850000 | 86900000 | 4050000 |
| 5 | chr10 | 10250000 | 13850000 | 3600000 |
| 6 | chr11 | 21950000 | 24650000 | 2700000 |
| 7 | chr11 | 36950000 | 39350000 | 2400000 |
| 8 | chr11 | 64800000 | 67950000 | 3150000 |
| 9 | chr11 | 79050000 | 84300000 | 5250000 |
| 10 | chr12 | 17050000 | 22550000 | 5500000 |
| 11 | chr13 | 7.00E+005 | 13250000 | 12550000 |
| 12 | chr13 | 15650000 | 29500000 | 13850000 |
| 13 | chr14 | 3950000 | 7700000 | 3750000 |
| 14 | chr14 | 20300000 | 24050000 | 3750000 |
| 15 | chr14 | 46650000 | 51150000 | 4500000 |
| 16 | chr14 | 97650000 | 102500000 | 4850000 |
| 17 | chr15 | 5500000 | 7550000 | 2050000 |
| 18 | chr15 | 4.60E+007 | 49650000 | 3650000 |
| 19 | chr16 | 7.00E+006 | 9.00E+006 | 2000000 |
| 20 | chr17 | 15700000 | 21400000 | 5700000 |
| 21 | chr17 | 35550000 | 39350000 | 3800000 |
| 22 | chr17 | 52900000 | 55350000 | 2450000 |
| 23 | chr17 | 60850000 | 63300000 | 2450000 |
| 24 | chr18 | 1.10E+007 | 13350000 | 2350000 |
| 25 | chr19 | 22250000 | 27950000 | 5700000 |
| 26 | chr2 | 6900000 | 8900000 | 2000000 |
| 27 | chr2 | 22150000 | 24350000 | 2200000 |
| 28 | chr2 | 84600000 | 88300000 | 3700000 |
| 29 | chr2 | 189100000 | 191700000 | 2600000 |
| 30 | chr20 | 50000 | 5450000 | 5400000 |
| 31 | chr20 | 50600000 | 54150000 | 3550000 |
| 32 | chr4 | 184600000 | 187550000 | 2950000 |
| 33 | chr5 | 8.00E+005 | 2900000 | 2100000 |
| 34 | chr5 | 4850000 | 7350000 | 2500000 |
| 35 | chr5 | 76600000 | 80900000 | 4300000 |
| 36 | chr6 | 8950000 | 12650000 | 3700000 |
| 37 | chr6 | 17200000 | 20100000 | 2900000 |
| 38 | chr6 | 101650000 | 106150000 | 4500000 |
| 39 | chr7 | 2400000 | 1.00E+007 | 7600000 |
| 40 | chr7 | 11250000 | 19850000 | 8600000 |
| 41 | chr8 | 17350000 | 20600000 | 3250000 |
| 42 | chr8 | 34100000 | 38900000 | 4800000 |
| 43 | chr8 | 81300000 | 83600000 | 2300000 |

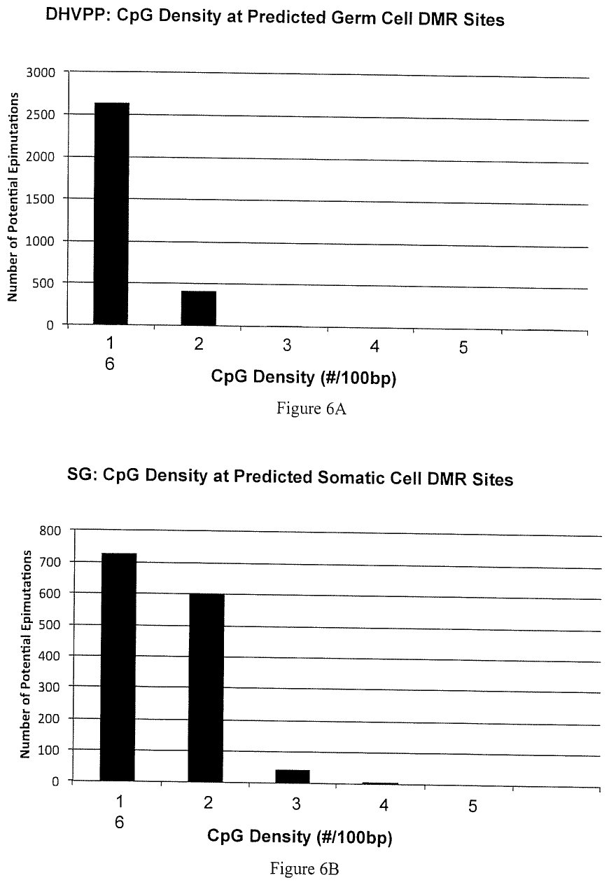

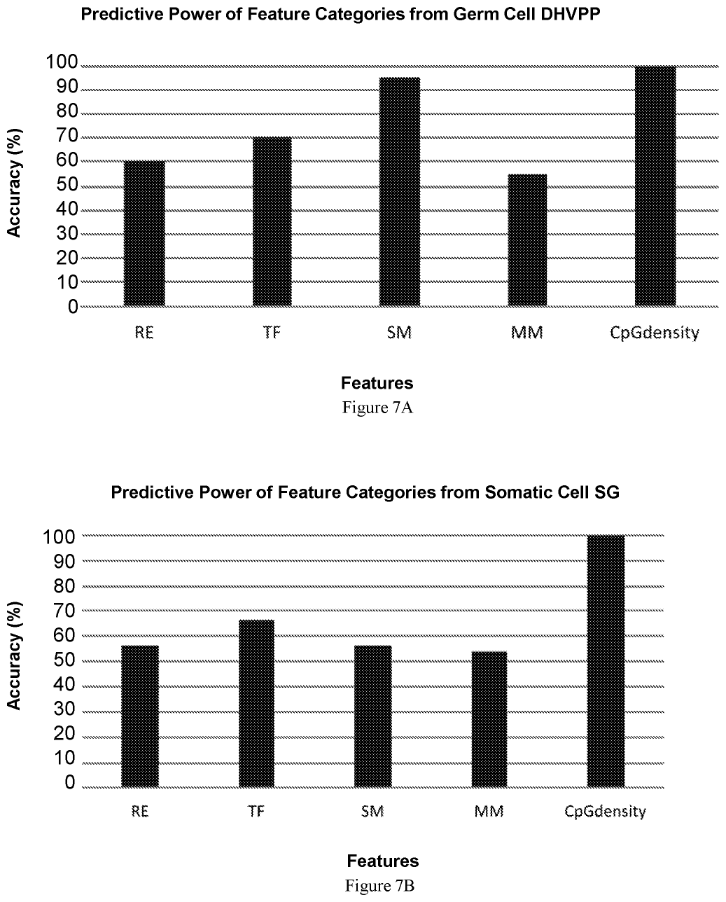

The following analyses investigated the genomic features of the predicted DMR/epimutations. The initial analysis was to check the CpG density of the regions which were identified as potential DMRs. The predicted DMR CpG density (number of CpG in each 100 bases) distribution was determined and shown in FIG. 6A-B. Interestingly, all the predicted DMR sites had densities of <2CpG/100 bp. This observation supports the fact that most DMRs are found in low CpG density regions (termed CpG deserts) [22] instead of regions of high CpG density (called CpG islands or shores) [36]. Prediction power refers to the number of DMR that contain a specific feature. The percentage of predicted DMR that had the CpG density feature (i.e., prediction power) was 100% for both the germ cell and somatic cell predicted DMR data sets (FIG. 7A-B).

Transcription factor binding sequence motifs and mammalian sequence motifs were the next features investigated. These features were collected from the DMR region and upstream and downstream of the DMR. Features were extracted from 1 k, 5 k and 100 k upstream and downstream regions of the DMR region. The consensus sequence correlations to the prediction of DMRs are shown in FIG. 7. For the predicted DMR in DHVPP that had the sequence motif features, the prediction power was high (above 90%) while for SG the prediction power of transcription factor features was above 60%. This was compared to the 100% predictive ability of CpG density.

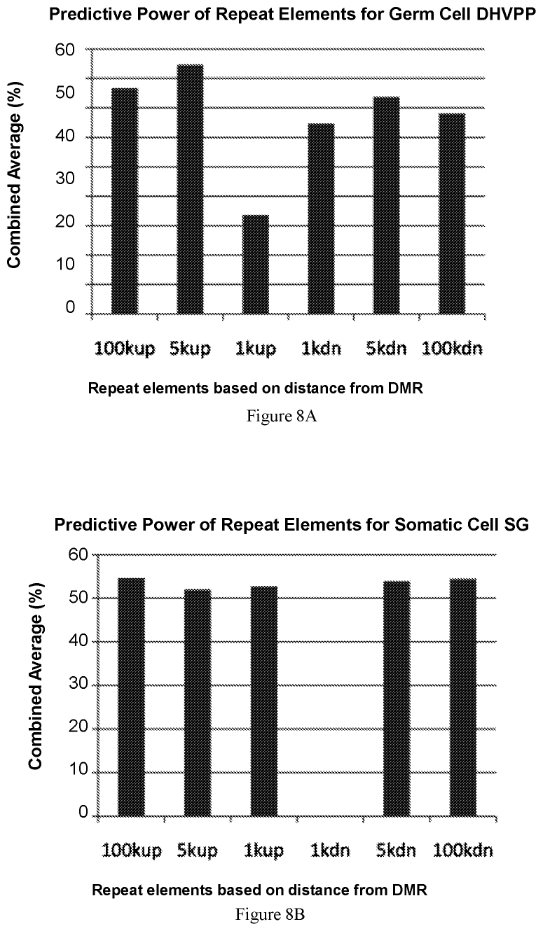

The repeat elements were chosen as a group of features (based on their location and distance from the DMR region) and for the predicted DMR that had the feature, prediction power was calculated to see which repeat elements gave the highest accuracy. All the repeat elements were grouped into 1 k, 5 k, 100 k upstream and downstream. The predictive power of repeat elements for DHVPP and SG is shown in FIG. 8A-B. The repeat elements in the 100 k upstream region had a slightly higher predictive power for the SG dataset. The repeat elements in the 5 k upstream had higher predictive power among the germ cell groups. The average DMR sites for DHVPP had a 3564 base length and for SG had a 4213 base length. The details are given in Tables 5 and 6.

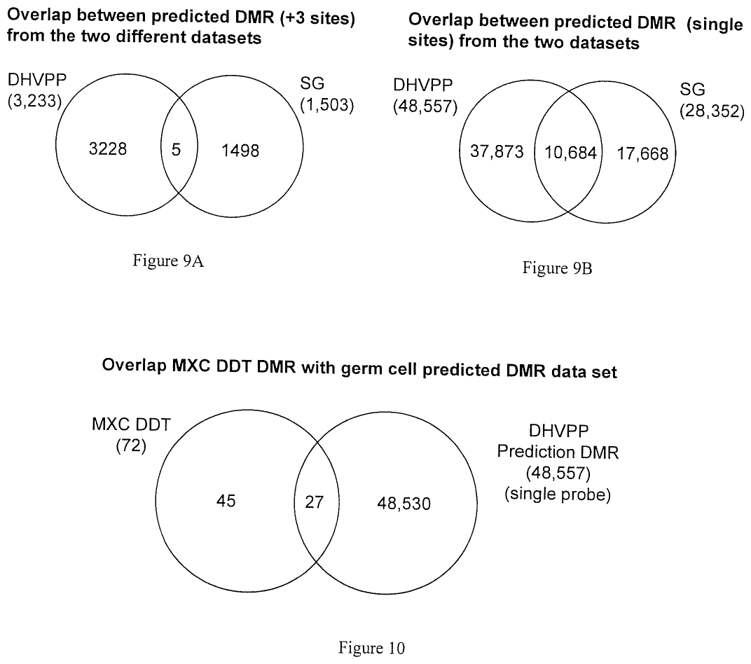

A comparison was made between the genome-wide predicted DMR/epimutation in the germ cell data sets and somatic cell data sets. The distribution of the predicted DMR on the various chromosomes is shown in Tables 9 and 10. Overlap between the potential predicted DMR sets derived from the germline DHVPP and somatic SG datasets showed only five common predicted sites (FIG. 9A). In addition, the overlap with the single predicted DMR sites identified 10K sites with overlap (FIG. 9B). This shows that the germline (sperm) predicted DMR and somatic (SG) cells predicted DMR are generally distinct. The sperm and somatic cell predicted DMR were obtained with different feature sets and independently. Therefore, the learned classifiers from the germline (DHVPP) and the somatic cell (SC) datasets are also distinct. This corresponds to the differences in contributions in the various genomic features. Since the original DMR somatic SG and germline DHVPP DMR sites had no overlap between them in the training data, it was not surprising very little overlap was observed among the predicted DMRs. These overlapped sites are shown in Venn diagrams in FIGS. 9A and B.

TABLE 9
Genomic chromosome locations of predicted DMR.
(A) Germ cell DHVPP and somatic cell SG predicted
number of (+3) sites in each chromosome.

| | DHVPP | SG |
|---|---|---|
| chr1 | 282 | 144 |
| chr2 | 371 | 141 |
| chr3 | 176 | 81 |
| chr4 | 129 | 87 |
| chr5 | 189 | 99 |
| chr6 | 172 | 94 |
| chr7 | 200 | 96 |
| chr8 | 142 | 82 |
| chr9 | 172 | 51 |
| chr10 | 51 | 45 |
| chr11 | 130 | 56 |
| chr12 | 72 | 23 |
| chr13 | 128 | 101 |
| chr14 | 163 | 67 |
| chr15 | 160 | 68 |
| chr16 | 138 | 46 |
| chr17 | 217 | 67 |
| chr18 | 120 | 47 |
| chr19 | 53 | 28 |
| chr20 | 48 | 30 |
| chrX | 120 | 50 |

TABLE 10
Genomic chromosome locations of predicted DMR.
(B) Germ cell DHVPP and somatic cell SG predicted
number of single sites in each chromosome.

| | DHVPP | SG |
|---|---|---|
| chr1 | 3780 | 2926 |
| chr2 | 5751 | 2820 |
| chr3 | 2642 | 1662 |
| chr4 | 4356 | 1935 |
| chr5 | 2569 | 1524 |
| chr6 | 2673 | 1560 |
| chr7 | 2483 | 1593 |
| chr8 | 2036 | 1332 |
| chr9 | 1952 | 1372 |
| chr10 | 613 | 1063 |
| chr11 | 1778 | 980 |
| chr12 | 291 | 226 |
| chr13 | 2035 | 1381 |
| chr14 | 2241 | 1211 |
| chr15 | 1985 | 1198 |
| chr16 | 1751 | 1003 |
| chr17 | 1882 | 1238 |
| chr18 | 1567 | 911 |
| chr19 | 618 | 642 |
| chr20 | 289 | 381 |
| chrX | 5265 | 1394 |

In order to help validate the machine learning results for the predicted germ cell DMR data set a positive validation analysis was performed. For the positive validation analysis the predicted DMR datasets were compared to two more recently developed sperm DMR datasets which were not used as test sets in the machine learning analysis. The first was a DDT transgenerational sperm DMR set [14] and second a methoxychlor (MXC) data set [13]. The two DMR positive control data sets were combined and termed the sperm MXC-DDT DMR data set. The description of the datasets is given in Table 3. The germ cell learned classifier accurately predicted all the DMRs in the sperm MXC-DDT dataset, 100% prediction accuracy (Table 9). Prediction accuracy is defined as the number of previously identified DMR that were identified by the computational tool. In addition, a comparison of the MXC-DDT DMR with the predicted genome-wide sperm DMR showed 38% overlap with the single site comparison (FIG. 10). Therefore, this positive validation sperm transgenerational DMR dataset was accurately predicted and had partial overlap, helping to validate the approach and predicted germ cell DMR dataset. Alternately, a negative validation analysis used a negative non-DMR (nDMR) data set involving transcription factor binding sites for SOX9, SRY and TCF21 [37,38] termed Sox9SryTcf21 with a total of 297 nDMR. This negative dataset was obtained with similar technology as the DMR sets. This involved a chromatin immunoprecipitation (ChIP) followed by a promoter tiling array (ChIP-Chip) analysis for this nDMR set versus the methylated DNA immunoprecipitation (MeDIP) followed by the tiling array (MeDIP-Chip). Using the negative nDMR data set and the machine learning algorithm only a 47% prediction accuracy (SG) and 42% prediction accuracy (DHVPP) was obtained while predicting all nDMR in Sox9SryTcf21 dataset (Table 9). A prediction accuracy of 50% or less is neutral with no prediction potential. Therefore, the negative validation with the nDMR demonstrated negligible overlap with the predicted DMR dataset and poor accuracy in the machine learning analysis.

TABLE 9
Validation of the germ cell DMR data set. MXC-DDT used as positive
testing set and Sox9SryTcf21 as non-DMR negative testing set.
Prediction of the training set DHVPP with the positive MXC-
DDT and negative Sx9SryTcf21 validation data set.

| | (Positive) | | (Negative) | |
|---|---|---|---|---|
| | MXC DDT | | Sox9SryTcf21 | |
| Training Set | (76 DMR) | Accuracy | (297 nDMR) | Accuracy |
| DHVPP | Predicted as | 100% | Predicted as | 42% |
| | 76 DMR | | 126 nDMR | |
| | | | (171 DMR) | |

### Discussion

Previous studies have demonstrated a variety of environmental factors from abnormal nutrition [39-45] to toxicant exposures can promote the epigenetic transgenerational inheritance of disease susceptibility and germline (e.g., sperm) epimutations [1]. Examples include the agricultural fungicide vinclozolin [11,17], the industrial contaminant dioxin [46,47], a hydrocarbon mixture jet fuel (JP8) [16], the plastic derived compounds bisphenol A (BPA) and phthalates [15,48,49], the pesticides methoxychlor [11,13] and dichlorodiphenyltrichloroethane (DDT) [14], and permethrin and N,N-Diethyl-meta-toluamide (DEET) [12]. All these environmental exposures of a gestating female (F0 generation) during the period of fetal gonadal sex determination promoted the epigenetic transgenerational (i.e. F3 generation) inheritance of disease. The transgenerational disease observed varied between the exposures, but generally involved abnormalities in the testis (spermatogenic cell apoptosis), ovary (polycystic ovarian disease), kidney (cyst development), prostate (epithelial cell atrophy), and behavioral abnormalities including mate preference changes and anxiety [1]. Interestingly, the chromosomal locations of the transgenerational sperm epimutations were generally distinct between the different exposure lineages [18]. Therefore, the sperm were found to have an exposure specific set of epimutations [1] and the epimutations all had common genomic features of a low CpG (<10 CpG/100 bp) density (i.e., CpG deserts) [22] and unique DNA sequence motifs [23].

The current study was designed to use these various transgenerational epimutation datasets as training sets in a novel sequential machine learning approach to identify the potential genome-wide locations of transgenerational epimutations. Although previous machine learning approaches applied active learning or imbalance class learning independently, the sequential use for a biological data set is novel. The training datasets from the epigenetic transgenerational (F3 generation) inheritance of sperm epimutations from various exposure lineages included; dioxin [46], jet fuel [16], vinclozolin [16,18,19,46], plastics (BPA phthalates) [15] and pesticide (permethrin and DEET) [12,15]. These exposure specific sperm epimutation datasets were used to develop the machine learning algorithm to predict the genome-wide locations of sperm epimutations. In addition, transgenerational somatic cell epimutation datasets were used to predict genome-wide locations of potential somatic epimutations. The testicular Sertoli cell and ovarian granulosa cells were purified from adult vinclozolin lineage F3 generation tissues and these cell specific epimutations identified [20,21]. These transgenerational somatic cells epimutation datasets were then used independently as training sets in the machine learning approach to develop the algorithm for transgenerational somatic cell epimutations and compare to that of transgenerational germline epimutation predictions.

In a previous research study that looked into finding potential imprinted genes in human and mouse genomes, Jirtle and colleagues mined the mouse genome and found thousands of relevant features for machine learning prediction of potential imprinted genes [25]. Imprinted genes are parent of origin monoallelic expressed genes with critical developmental functions [50]. Mining the DNA sequence characteristics up to 100 kb upstream and downstream around known imprinted genes developed genomic features and training sets to develop a prediction algorithm [25]. They used the Equbits Foresight (www.equbits.com) classifier and predicted 722 new potential imprinted gene sites. Their study examined 23,788 annotated autosomal mouse genes and identified 600 potential mouse imprinted genes [25]. The same group later mined the human genome for new imprinted sites [26]. They again used the Equbits Foresight which uses the Support Vector Machine (SVM) classifier and 622 features and used their own SMLR (sparse multinomial logistic regression) [51] classifier with 820 features to predict novel human imprinted genes [26]. A second study by another group looked into the correlation of different genomic features in DNA methylation of CpG islands [52]. They mined features from 190 CpG islands from human chromosome 21 and tested it on the rest of the CpG islands in the genome for finding potential methylated CpG islands. A correlation among different features identified potential different methylation profiles for different tissue types and for different diseases [52]. The main difference of the proposed approach with the imprinted gene research is that active learning is used to identify a sub-group of features for each queried training example instead of using a global feature reduction [25,26]. For the second study, the main difference is that their approach looks into DNA methylation in CpG islands while the current study looks into genome wide methylation patterns including low density CpG regions, unlike dense CpG regions in CpG islands [52].

Active learning using the GQAL approach on the transgenerational sperm DHVPP epimutation was done over a 10 fold cross validation. During training GQAL found 36% of the features to be redundant and used 245 samples averaged over all iterations. Once training was complete the learning algorithm was tested on an independent test set and an accuracy of 99.2% was achieved. In contrast, for the somatic cell (SC) dataset GQAL removed 14% of the features as redundant and used 290 samples averaged over all iterations. Again after completion of training the learned classifier was tested on an independent test set and achieved an accuracy of 97.7%. This shows the power of the GQAL approach [30]. While Active Learning removes redundant features, boosting performed balanced learning on the epigenetic datasets.

Additional analysis was done to determine the predictive power of specific groups and individual genomic features. The percentage of predicted DMR that contained a feature was used for “prediction power”. For the final prediction the combined groups of features had the highest impact with 100% accuracy compared to individual features. As observed for individual features, FIG. 7, it can be seen that SG transcription factors have above 60% prediction power which is not that high compared to the neutral impact of 50%. However, DHVPP sequence motifs have over 90% power followed by 70% for transcription factors. When only single features are used for training, their power of prediction is generally lower than when combined. For both datasets, CpG density had a high prediction power rate of 99%. For DHVPP, a number of features for example, MOTIF CCGG and GCGC have higher than 90% prediction power, followed by TCGG which has higher than 80% prediction power. All of these motifs were constructed by running the predicted initial DMR sites through a number of motif finding algorithms to find new motif sequences which were used for prediction [23]. Among those highly selected motifs these few performed well and were chosen for the final 134 features for DHVPP sperm dataset.

Once the two step training was completed the trained model was used for a genome-wide prediction. The rat genome was annotated with all the genomic features selected and the learned classifier was applied. Among the initial list of predicted 48K sites for the sperm DHVPP and 28K for somatic SG sites, after selecting only the three or more consecutive sites a final list of 3,233 sites for DHVPP germline cell and 1,502 sites for somatic cell SG remained. There are more sites in the DHVPP in part since this is a combination of five different experiments. In contrast, somatic cell SG datasets involved two individual cell types from the testis and ovaries only and the number of epimutations was less than the germ cell datasets.

The number of specific DMR that localized onto each chromosome for the somatic cell 1,502 sites and germ cell 3,233 sites was found to be comparable between chromosomes (Table 9). Chromosome 1 and 2 for both datasets show higher numbers of sites in part due to the size of these chromosomes. A cluster analysis for genomic regions with a statistically significant over-representation of predicted DMR identified a number of clusters on each chromosome (FIGS. 4 and 5). Previously over-represented differential gene expression near DMR were identified as Epigenetic Control Regions (ECR) [35], similar to Imprinting Control Regions (ICR) [62]. The speculation is these clustered DMR have a role in the epigenetic regulation of gene expression in large regions of 2-5 megabases (Tables 7 and 8) [35].

Interestingly, the predicted germ cell DMR and somatic DMR were distinct with negligible overlap (FIG. 9A-B). In addition, the leamed classifiers and the critical genomic features were also different between germ cell and somatic cell DMR. However, the CpG desert feature was common between the predicted DMR datasets. Observations suggest the molecular elements and characteristics of the somatic cell and germ cell DMR are distinct. As different feature sets were used for training for both germ cells and somatic cells, the predicted DMR have negligible overlap. Although the CpG density was common and critical for both, the other features were more variable. Since the germ cell DMR are important for the epigenetic transgenerational inheritance of disease and phenotypic variation [1], while the somatic cell DMR are relevant to the gene regulation with specific cell types, it is not surprising that the molecular characteristic of the DMR are distinct.

A partial validation of the novel machine learning approach and predicted genome-wide germ cell DMR used recently identified sperm DMR not used as training data sets. The transgenerational sperm epimutations from DDT [14] and methoxychlor [13] lineage F3 generation animals were combined and used as a positive validation DMR data set termed MXC-DDT. Since these are independently identified transgenerational sperm DMR, they should appear in the transgenerational machine learning predicted genome-wide sperm DMR data set. The analysis showed 100% prediction accuracy of the MXC-DDT DMR being selected by the machine learning algorithm when used as a training set. The MXC-DDT DMR were found to have a 38% overlap with the single sites in comparison with the predicted sperm DMR dataset (FIG. 10). This observation helps validate the machine learning approach and predicted genome-wide datasets obtained. In contrast, a negative validation data set used a set of transcription factor binding sites that are irrelevant to DMR and had negligible overlap nor selection. For example, the negative validation data set sites generally had high density CpG (less than 42% had low density CpG sites). Although clearly identified non-DMR data sets are difficult to obtain, this negative validation data set used helps support the prediction power and accuracy of the current study.

### CONCLUSION

The novel machine learning approach utilized a sequential generalized query based active learning and imbalance class learning on epigenetic data sets. Some studies have applied machine learning to epigenetics [25,26]. However, the machine learning approach developed can be used to increase the accuracy and efficiency of the prediction of machine learning with any biological dataset or any dataset for that matter. The advantage to this novel sequential machine learning approach is better accuracy through balancing the datasets and then using optimal features to train the classifier and increase efficiency. The current approach used a tandem sequential process, but the the active and imbalance learning can be combined into a single process. Broader use of this approach is anticipated to improve the specific machine learning tool developed and enhance machine learning applications.

A variety of different environmental exposures [1] have been shown to induce the epigenetic inheritance of disease and phenotypic variation in species ranging from plants, flies, worms, fish, rodents, pigs and humans [1,11,43,63-67]. The germline transmission of altered epigenetic information is the mechanism behind this non-genetic form of inheritance [9]. Differential DNA methylated regions (DMRs) are in part the epigenetic mechanism of epigenetic inheritance [1]. Previous studies have demonstrated the DMRs termed epimutations identified are exposure specific [18] and correlate to later life disease susceptibility [1]. A variety of different disease conditions, behavioral alterations and phenotypic variation is associated with the epigenetic transgenerational inheritance phenomenon [1]. Identification of DMR or epimutations associated with ancestral or early life exposures correlates to later life disease [18]. A number of studies have demonstrated the feasibility of these epigenetic biomarkers that could be used as early stage diagnostics for disease susceptibility [1]. The current study used a novel sequential machine learning approach to predict the potential susceptible DMR and epimutation sites in the genome. This information and datasets can now be used to more effectively identify the patterns or signatures of DMR associated with specific exposures and disease conditions.

In addition to the prediction of the genome-wide DMR and potential epimutations, the novel machine learning tool also provides critical information regarding the essential genomic molecular features of the DMR. The most important was the low density CpG regions or CpG deserts (FIG. 6). The evolutionary significance and regulatory role of such regions has been previously discussed [8,22]. The assumption is the genomic features identified will be highly conserved among species, in particular mammals. Therefore, the developed machine learning tool may be applicable to many species including humans. The tool may provide a predicted DMR dataset that can be used to facilitate human epigenetic biomarker identification. Therefore, the observations have provided a useful new machine learning approach and tool for computational biology. In addition, valuable new molecular insights and datasets have been provided to help elucidate the environmentally induced epigenetic transgenerational inheritance phenomenon.

### REFERENCES

1. Skinner M K (2014) Endocrine disruptor induction of epigenetic transgenerational inheritance of disease. Mol Cell Endocrinol 398: 4-12.
2. Waddington C H (1953) Epigenetics and evolution. Symp Soc Exp Biol 7: 186-199
3. Skinner M K, Manikkam M, Guerrero-Bosagna C (2010) Epigenetic transgenerational actions of environmental factors in disease etiology. Trends Endocrinol Metab 21: 214-222.
4. Holliday R, Pugh JE (1975) DNA modification mechanisms and gene activity during development. Science 187: 226-232.
5. Singer J, Roberts-Ems J, Riggs AD (1979) Methylation of mouse liver DNA studied by means of the restriction enzymes msp I and hpa II. Science 203: 1019-1021.
6. Kornfeld J W, Bruning J C (2014) Regulation of metabolism by long, non-coding RNAs. Front Genet 5: 57.
7. Yaniv M (2014) Chromatin remodeling: from transcription to cancer. Cancer Genet 207: 352-357.
8. Skinner M K, Guerrero-Bosagna C, Haque M M, Nilsson E E, Koop J A H, et al. (2014) Epigenetics and the evolution of Darwin's Finches Genome Biology & Evolution 6: 1972-1989.
9. Skinner M K (2014) A new kind of inheritance. Sci Am 311: 44-51.
10. Dias B G, Maddox S A, Klengel T, Ressler K J (2014) Epigenetic mechanisms underlying learning and the inheritance of learned behaviors. Trends Neurosci.
11. Anway M D, Cupp A S, Uzumcu M, Skinner M K (2005) Epigenetic transgenerational actions of endocrine disruptors and male fertility. Science 308: 1466-1469.
12. Manikkam M, Tracey R, Guerrero-Bosagna C, Skinner M (2012) Pesticide and Insect Repellent Mixture (Permethrin and DEET) Induces Epigenetic Transgenerational Inheritance of Disease and Sperm Epimutations. Reproductive Toxicology 34: 708-719.
13. Manikkam M, M H M, Guerrero-Bosagna C, Nilsson E, Skinner M (2014) Pesticide methoxychlor promotes the epigenetic transgenerational inheritance of adult onset disease through the female germline. PLoS ONE 9: e102091.
14. Skinner M K, Manikkam M, Tracey R, Nilsson E, Haque M M, et al. (2013) Ancestral DDT Exposures Promote Epigenetic Transgenerational Inheritance of Obesity BMC Medicine 11: 228.
15. Manikkam M, Tracey R, Guerrero-Bosagna C, Skinner M (2013) Plastics Derived Endocrine Disruptors (BPA, DEHP and DBP) Induce Epigenetic Transgenerational Inheritance of Adult-Onset Disease and Sperm Epimutations. PLoS ONE 8: e55387.
16. Tracey R, Manikkam M, Guerrero-Bosagna C, Skinner M (2013) Hydrocarbon (Jet Fuel JP-8) Induces Epigenetic Transgenerational Inheritance of Adult-Onset Disease and Sperm Epimutations. Reproductive Toxicology 36: 104-116.
17. Anway M D, Leathers C, Skinner M K (2006) Endocrine disruptor vinclozolin induced epigenetic transgenerational adult-onset disease. Endocrinology 147: 5515-5523.
18. Manikkam M, Guerrero-Bosagna C, Tracey R, Haque M M, Skinner M K (2012) Transgenerational actions of environmental compounds on reproductive disease and identification of epigenetic biomarkers of ancestral exposures. PLoS ONE 7: e31901.
19. Guerrero-Bosagna C, Settles M, Lucker B, Skinner M (2010) Epigenetic transgenerational actions of vinclozolin on promoter regions of the sperm epigenome. Plos One 5: e13100.
20. Guerrero-Bosagna C, Savenkova M, Haque M M, Sadler-Riggleman I, Skinner MK (2013) Environmentally Induced Epigenetic Transgenerational Inheritance of Altered Sertoli Cell Transcriptome and Epigenome: Molecular Etiology of Male Infertility. PLoS ONE 8: e59922.
21. Nilsson E, Larsen G Manikkam M, Guerrero-Bosagna C, Savenkova M, et al. (2012) Environmentally Induced Epigenetic Transgenerational Inheritance of Ovarian Disease. PLoS ONE 7: e36129.
22. Skinner M K, Guerrero-Bosagna C (2014) Role of CpG Deserts in the Epigenetic Transgenerational Inheritance of Differential DNA Methylation Regions. BMC Genomics 15: 692.
23. Guerrero-Bosagna C, Weeks S, Skinner MK (2014) Identification of genomic features in environmentally induced epigenetic transgenerational inherited sperm epimutations. PLoS One 9: e100194.
24. Weber M, Schubeler D (2007) Genomic patterns of DNA methylation: targets and function of an epigenetic mark. Curr Opin Cell Biol 19: 273-280.
25. Luedi P P, Hartemink A J, Jirtle R L (2005) Genome-wide prediction of imprinted murine genes. Genome Res 15: 875-884.
26. Luedi P P, Dietrich F S, Weidman J R, Bosko J M, Jirtle R L, et al. (2007) Computational and experimental identification of novel human imprinted genes. Genome Res 17: 1723-1730.
27. Luger G (2009) Artificial Intelligence: Structures and Strategies for Complex Problem Solving (6th Edition): Addison-Wesley.
28. Lin W J, Chen J J (2013) Class-imbalanced classifiers for high-dimensional data. Brief Bioinform 14: 13-26.
29. Chen Y, Carroll R J, Hinz E R, Shah A, Eyler A E, et al. (2013) Applying active learning to high-throughput phenotyping algorithms for electronic health records data. J Am Med Inform Assoc 20: e253-259.
30. Haque M M, Holder L B, Skinner M K, Cook D J (2013) Generalized Query Based Active Learning to Identify Differentially Methylated Regions in DNA. IEEE/ACM Trans Comput Biol Bioinform 10: 632-644.
31. Haque M M, Skinner M K, Holder L B (2014) Imbalanced Class Learning in Epigenetics. Journal of Computational Biology 21: 492-507.
32. Sandelin A, Alkema W, Engstrom P, Wasserman W W, Lenhard B (2004) JASPAR: an open-access database for eukaryotic transcription factor binding profiles. Nucleic Acids Res 32: D91-94.
33. Das M K, Dai H K (2007) A survey of DNA motif finding algorithms. BMC Bioinformatics 8 Suppl 7: S21.
34. Stormo G D (2000) DNA binding sites: representation and discovery. Bioinformatics 16:16-23.
35. Skinner M K, Manikkam M, Haque M M, Zhang B, Savenkova M (2012) Epigenetic Transgenerational Inheritance of Somatic Transcriptomes and Epigenetic Control Regions. Genome Biol 13: R91
36. Illingworth R S, Bird A P (2009) CpG islands—‘a rough guide’. FEBS Lett 583: 1713-1720.
37. Bhandari R, Haque Md. M, Skinner M (2012) Global Genome Analysis of the Downstream Binding Targets of Testis Determining Factor SRY AND SOX9. PLoS ONE 7: e43380.
38. Bhandari R K, Schinke E N, Haque M M, Sadler-Riggleman I, Skinner MK (2012) SRY Induced TCF21 Genome-Wide Targets and Cascade of bHLH Factors During Sertoli Cell Differentiation and Male Sex Determination in Rats. Biol Reprod 87: 131.
39. Burdge G C, Slater-Jefferies J, Torrens C, Phillips E S, Hanson M A, et al. (2007) Dietary protein restriction of pregnant rats in the F0 generation induces altered methylation of hepatic gene promoters in the adult male offspring in the F1 and F2 generations. Br J Nutr 97: 435-439.
40. Burdge G C, Hoile S P, Uller T, Thomas N A, Gluckman P D, et al. (2011) Progressive, Transgenerational Changes in Offspring Phenotype and Epigenotype following Nutritional Transition. PLoS ONE 6: e28282.
41. Dunn G A, Morgan C P, Bale T L (2011) Sex-specificity in transgenerational epigenetic programming. Horm Behav 59: 290-295.
42. Painter R C, Osmond C, Gluckman P, Hanson M, Phillips D I, et al. (2008) Transgenerational effects of prenatal exposure to the Dutch famine on neonatal adiposity and health in later life. BJOG 115: 1243-1249.
43. Pembrey M E (2010) Male-line transgenerational responses in humans. Hum Fertil (Camb) 13: 268-271.
44. Pembrey M E, Bygren L O, Kaati, Edvinsson S, Northstone K, et al. (2006) Sex-specific, male-line transgenerational responses in humans. Eur J Hum Genet 14: 159-166.
45. Veenendaal M V, Painter R C, de Rooij S R, Bossuyt P M, van der Post J A, et al. (2013) Transgenerational effects of prenatal exposure to the 1944-45 Dutch famine. BJOG 120: 548-553.
46. Manikkam M, Tracey R, Guerrero-Bosagna C, Skinner M K (2012) Dioxin (TCDD) induces epigenetic transgenerational inheritance of adult onset disease and sperm epimutations. PLoS ONE 7: e46249.
47. Bruner-Tran K L, Osteen K G (2011) Developmental exposure to TCDD reduces fertility and negatively affects pregnancy outcomes across multiple generations. Reprod Toxicol 31: 344-350.
48. Salian S, Doshi T, Vanage G (2009) Impairment in protein expression profile of testicular steroid receptor coregulators in male rat offspring perinatally exposed to Bisphenol A. Life Sci 85: 11-18.
49. Wolstenholme J T, Goldsby J A, Rissman E F (2013) Transgenerational effects of prenatal bisphenol A on social recognition. Horm Behav 64: 833-839.
50. Barlow D P, Bartolomei M S (2014) Genomic imprinting in mammals. Cold Spring Harb Perspect Biol 6.
51. Krishnapuram B, Carin L, Figueiredo M A, Hartemink A J (2005) Sparse multinomial logistic regression: fast algorithms and generalization bounds. IEEE Trans Pattern Anal Mach Intell 27: 957-968.
52. Wrzodek C, Buchel F, Hinselmann G, Eichner J, Mittag F, et al. (2012) Linking the epigenome to the genome: correlation of different features to DNA methylation of CpG islands. PLoS ONE 7: e35327.
53. Settles B, Craven M (2008) An analysis of active learning strategies for sequence labeling tasks. Proceedings of Empirical Methods in Natural Language Processing, EMNLP '08: 1070-1079.
54. Lewis D D, Catlett J (1994) Heterogeneous uncertainty sampling for supervised learning. Proceedings of the International Conference on Machine Learning ICML'94: 148-156.
55. Holte R C, Acker L E, Porter B W (1989) Concept learning and the problem of small disjuncts. Proceedings of the Eleventh International Joint Conference on Artificial Intelligence. pp. 813-818.
56. Mease D, Wyner A J, Buja A (2007) Boosted classification trees and class probability/quantile estimation. The Journal of Machine Learning Research 8: 409-439.
57. Drummond C, Holte R C (2003) C4.5, class imbalance, and cost sensitivity: why under-sampling beats over-sampling. Workshop on Learning from Imbalanced Datasets II. pp. 1-8.
58. Schapire R E (1990) The strength of weak learnability Machine learning 5: 197-227.
59. Freund Y, Schapire R E (1995) A desicion-theoretic generalization of on-line learning and an application to boosting. In: Springer, editor. Computational learning theory. Berlin Heidelberg. pp. 23-37.
60. Friedman N, Geiger D, Goldszmidt M (1997) Bayesian Network Classifiers. Machine Learning 29: 131-163.
61. Bender A (2011) Bayesian methods in virtual screening and chemical biology. Methods Mol Biol 672: 175-196.
62. Wan L B, Bartolomei M S (2008) Regulation of imprinting in clusters: noncoding RNAs versus insulators. Adv Genet 61: 207-223.
63. Crevillen P, Yang H, Cui X, Greeff C, Trick M, et al. (2014) Epigenetic reprogramming that prevents transgenerational inheritance of the vemalized state. Nature 515: 587-590.
64. Xing Y, Shi S, Le L, Lee C A, Silver-Morse L, et al. (2007) Evidence for transgenerational transmission of epigenetic tumor susceptibility in Drosophila. PLoS Genet 3: 1598-1606.
65. Kelly W G (2014) Multigenerational chromatin marks: no enzymes need apply. Dev Cell 31: 142-144.
66. Baker T R, Peterson R E, Heideman W (2014) Using Zebrafish as a Model System for Studying the Transgenerational Effects of Dioxin. Toxicol Sci 138: 403-411.
67. Braunschweig M, Jagannathan V, Gutzwiller A, Bee G (2012) Investigations on transgenerational epigenetic response down the male line in F2 pigs. PLoS ONE 7:e30583.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A method comprising:
(a) obtaining sequencing data from a plurality of nucleotide sequences obtained from a sample from a person having a condition or suspect of having said condition, wherein a nucleotide sequence of the plurality of nucleotide sequences comprises a first plurality of genomic features and one or more differential DNA methylation regions (DMRs);
(b) iteratively training an algorithm, using one or more computer processors, with said sequencing data to obtain a trained algorithm with a second plurality of genomic features, wherein said algorithm comprises active learning (AL); and
(c) applying the trained algorithm to at least a portion of a nucleotide sequence to identify a DMR.

2. The method of claim 1, further comprising outputting a report containing said DMR in (c) and said condition.

3. The method of claim 1, wherein said first plurality of genomic features comprises CpG density information, repeat elements, transcription factor response elements, sequence motifs, and mammalian genomic sequence motifs.

4. The method of claim 1, wherein said sample comprises a plurality of germline cells.

5. The method of claim 4, wherein said second plurality of genomic features is listed in Table 1.

6. The method of claim 4, wherein said second plurality of genomic features is categorized in Table 5.

7. The method of claim 4, wherein said DMR in (c) is listed in Table 7.

8. The method of claim 1, wherein said sample comprises a plurality of somatic cells.

9. The method of claim 8, wherein said second plurality of genomic features is listed in Table 2.

10. The method of claim 8, wherein said second plurality of genomic features is categorized in Table 6.

11. The method of claim 8, wherein said DMR in (c) is listed in Table 8.

12. The method of claim 1, wherein said AL is Generalized Query Bases Active Learning (GQAL).

13. The method of claim 12, wherein said GQAL comprises a base classifier.

14. The method of claim 13, where said base classifier is Tree Augmented Naive Bayes (TAN).

15. The method of claim 1, wherein said algorithm comprises an imbalanced class learning (ICL).

16. The method of claim 15, wherein said ICL comprises Adaboost.

17. The method of claim 1, wherein said DMR in (c) comprises a CpG density of fewer than 3 CpG per 100 base pairs.

18. The method of claim 1, wherein said condition is an exposure to a chemical.

19. The method of claim 18, wherein said chemical is selected from the group consisting of a bisphenol A (BPA), bis(2-ethylhexyl)phthalate (DEHP), dibutyl phthalate (DBP), N, N-diethyl-meta-toluamide (DEET), dichlorodiphenyltrichloroethane (DDT), pyrethroid, polychlorinated dibenzodioxin, hydrocarbon mixture, and any combination thereof.

20. The method of claim 1, wherein said condition comprises a severity level.

21. The method of claim 1, further comprising providing a therapeutic intervention to said subject for said condition.

22. The method of claim 21, further comprising monitoring a response to said therapeutic intervention in said subject.

23. The method of claim 1, further comprising sequencing said at least a portion of said nucleotide sequence in (c).


SEQUENCE LISTING (2 sequences)

SEQ ID NO: 1 - DNA, 36 bp, Artificial Sequence
  Synthetic transcription factor
atttttttatttttattttattttttttttttaaaa

SEQ ID NO: 2 - DNA, 24 bp, Artificial Sequence
  Synthetic motif
tggaggggcagtccggctcctggg